(19) United States
(12) Patent Application Publication
(10) Pub. No.: US 2016/0380203 A1
(43) Pub. Date: Dec. 29, 2016

(54) DIBENZOSUBERANE-BASED ELECTRON-TRANSPORT MATERIALS

(71) Applicants: Samson A. Jenekhe, Seattle, WA (US); Soonok Jeon, Seattle, WA (US); Taeshik Earmme, Seattle, WA (US); UNIVERSITY OF WASHINGTON, Seattle, WA (US)

(72) Inventors: Samson A. Jenekhe, Seattle, WA (US); Soonok Jeon, Seattle, WA (US); Taeshik Earmme, Seattle, WA (US)

(21) Appl. No.: 15/109,250

(22) PCT Filed: Dec. 30, 2014
(86) PCT No.: PCT/US2014/072685

§ 371 Date: Jun. 30, 2016

Related U.S. Application Data
(60) Provisional application No. 61/922,202, filed on Dec. 31, 2013.

(51) Int. Cl. H01L 51/00; C07D 217/02; C07C 25/22; C07C 17/23; C07C 1/32; C09K 11/02; C09K 11/06; C09D 11/52; C07C 13/547; C07D 213/06; C07C 17/263

(52) U.S. Cl. CPC H01L 51/0056; C07D 213/06; C07D 217/02; C07C 25/22; C07C 17/23; C07C 17/2637; C07C 17/263; H01L 51/0067; H01L 51/0072; C09K 11/025; H01L 51/007; H01L 51/0042; H01L 51/0037; H01L 51/0085; C09K 11/06; C09D 11/52; C07C 13/547; C07C 1/321; C07C 2103/94; H01L 2051/0063; C09K 2211/1416; C09K 2211/185; C07C 2531/12; H01L 51/5072

(57) ABSTRACT

Novel dibenzosuberane-based compounds, compositions containing such compounds, and electronic devices containing such compounds as electron transport materials are described herein. Methods for making the dibenzosuberane-based compounds of the present invention are also described.

### CROSS REFERENCE TO RELATED APPLICATIONS

This application claims the priority of U.S. Provisional Application No. 61/922,202 filed Dec. 31, 2013, which is hereby incorporated by reference in its entirety.

### FIELD OF THE INVENTION

The present invention relates to novel dibenzosuberane-based compounds and electronic devices containing such compounds as electron transport materials.

### BACKGROUND

Organic light-emitting diodes (OLEDs) are an important feature in modern display and lighting technologies, such as, for example, full-color flat displays, flexible displays, and solid-state lighting. Phosphorescent organic light-emitting diodes (PhOLEDs), an important class of OLEDs, are theoretically capable of achieving a 100% internal quantum efficiency by fully harvesting both singlet and triplet excitons. Therefore, PhOLEDs have attracted much attention for their applications in full-color displays and lighting. One promising strategy to obtain highly efficient PhOLEDs is to utilize high triplet energy materials to confine triplet excitons inside an emission layer (EML) in multilayered device structures.

High triplet energy materials are mainly used in EMLs as a host material or in adjacent hole transport layers (HTL) and electron transport layers (ETL). Use of high triplet energy confines triplet excitons inside the EML and suppresses triplet exciton quenching. In multilayered PhOLEDs, the ETL plays an important role in facilitating electron-injection/transport from a cathode while also acting as efficient exciton blocker. It is therefore preferable that the ETL have good electron-transport property, wide energy gap and high triplet energy. A highest occupied molecular orbital (HOMO) level of the electron-transport material is preferably deep enough to block hole carrier leakage and a lowest unoccupied molecular orbital (LUMO) level is preferably low enough to enable efficient electron injection from the cathode. Electron transport materials with high triplet energy preferably exhibit electrochemical, photochemical, and morphological stability.

Various electron-transport materials (ETMs) such as pyridine, phenylpyrimidine, triazine, quinoline, and phosphine oxide (PO) derivatives have been mainly used to achieve high-performance PhOLEDs. Dibenzothiophene-S,S-dioxide and thiophene-S,S-dioxide oligomers and polymers have not been usually viewed as suitable ETMs for PhOLED devices. Although they function as good ETMs for devices with high electron mobilities (10⁻⁴-10⁻³ cm²V⁻¹s⁻¹), their low band gap and low triplet energy are in many cases not suitable for efficient PhOLEDs, especially for a blue triplet emitter with high triplet energy.

There is a continuing, unresolved interest in developing materials having good electron-transport properties, wide energy gap, and/or high triplet energy.

### SUMMARY OF THE INVENTION

In a first aspect, the present invention is directed to compounds having the structure represented by formula (I):

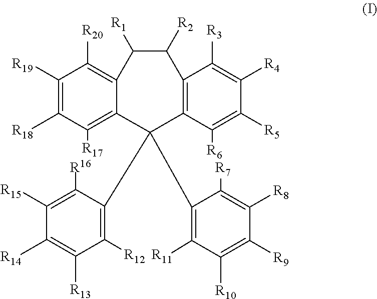

wherein R₁-R₂₀ are as defined herein.

In a second aspect, the present invention is directed to methods of making compounds having the structure represented by formula (I).

In a third aspect, the present invention is directed to compositions comprising compounds having the structure represented by formula (I).

In a fourth aspect, the present invention is directed to uses of a compound having the structure represented by formula (I).

The compounds according to the present invention exhibit high triplet energy as well as good electron transport properties.

### BRIEF DESCRIPTION OF THE DRAWINGS

FIG. 1 shows a schematic diagram of an electronic device according to the present invention.

FIG. 2 shows molecular structures and calculated HOMO/LUMO orbitals of dibenzosuberane-based compounds according to the present invention.

FIG. 3 shows the normalized absorption and PL emission spectra of (a) 2PySDP (square); (b) 3PySDP (circle); (c) 4PySDP (triangle); and (d) PSDP (inverse triangle).

FIG. 4 shows the normalized phosphorescence spectra of dibenzosuberane-based compounds at 77 K: (a) 2PySDP; (b) 3PySDP; (c) 4PySDP; and (d) PSDP.

FIG. 5 shows the TGA and DSC thermograms of (a),(e) 2PySDP; (b),(f) 3PySDP; (c),(g) 4PySDP; and (d),(h) PSDP.

FIG. 6 shows the normalized absorption and PL emission spectra of (a) 3DPySDP; (b) 4DPySDP; and (c) DPSDP.

FIG. 7 shows the normalized phosphorescence spectra of dibenzosuberane-based compounds at 77 K: (a) 3DPySDP; (b) 4DPySDP; and (c) DPSDP.

FIG. 8 shows the TGA thermograms of (a) 3DPySDP; (b) 4DPySDP; and (c) DPSDP.

FIG. 9 shows the DSC thermograms of (a) 3DPySDP; (b) 4DPySDP; and (c) DPSDP.

FIG. 10 shows the cyclic voltammograms of (a) 3DPySDP; (b) 4DPySDP; and (c) DPSDP.

FIG. 11 shows the normalized absorption and PL emission spectra of dibenzosuberane-based compounds in dilute THF solution (10⁻⁵M) and in thin films: (a) 2,7-DPySDF and (b) 3,6-DPySDF.

FIG. 12 shows the normalized phosphorescence spectra of dibenzosuberane-based compounds at 77 K: (a) 2,7-DPySDF and (b) 3,6-DPySDF.

FIG. 13 shows the TGA thermograms of (a) 2,7-DPySDF, and (b) 3,6-DpySDF.

FIG. 14 shows the DSC thermograms of (a) 2,7-DPySDF, and (b) 3,6-DpySDF.

FIG. 15 shows the cyclic voltammograms of (a) 2,7-DPySDF and (b) 3,6-DPySDF.

FIG. 16 shows the current density-voltage (J-V) characteristics of the blue PhOLEDs according to the present invention in (a) log-scale and (b) linear scale.

FIG. 17 shows the luminance-voltage (L-V) characteristics of the blue PhOLEDs according to the present invention in (a) log-scale and (b) linear scale.

FIG. 18 shows the (a) luminous efficiency-luminance (LE-L) and (b) power efficiency-luminance (PE-L) characteristics of the blue PhOLEDs according to the present invention.

FIG. 19 shows the cyclic voltammograms of (a) 2PySDP, (b) 3PySDP, (c) 4PySDP, and (d) PSDP.

### DETAILED DESCRIPTION OF THE INVENTION

As used herein, the following terms have the meanings ascribed below:
“anode” means an electrode that is more efficient for injecting holes compared to than a given cathode,
“buffer layer” generically refers to electrically conductive or semiconductive materials or structures that have at least one function in an electronic device, including but not limited to, planarization of an adjacent structure in the device, such as an underlying layer, charge transport and/or charge injection properties, scavenging of impurities such as oxygen or metal ions, and other aspects to facilitate or to improve the performance of the electronic device,
“cathode” means an electrode that is particularly efficient for injecting electrons or negative charge carriers,
“electroactive” when used herein in reference to a material or structure, means that the material or structure exhibits electronic or electro-radiative properties, such as emitting radiation or exhibiting a change in concentration of electron-hole pairs when receiving radiation,
“electronic device” means a device that comprises one or more layers comprising one or more semiconductor materials and makes use of the controlled motion of electrons through the one or more layers,
“electron injection” or “electron transport”, as used herein in reference to a material or structure, means that such material or structure that promotes or facilitates migration of negative charges through such material or structure into another material or structure,
“hole injection” or “hole transport” when used herein when referring to a material or structure, means such material or structure facilitates migration of positive charges through the thickness of such material or structure with relative efficiency and small loss of charge,
“layer” as used herein in reference to an electronic device, means a coating covering a desired area of the device, wherein the area is not limited by size, that is, the area covered by the layer can, for example, be as large as an entire device, be as large as a specific functional area of the device.

As used herein, the terminology “(Cₓ-C_{y})” in reference to an organic group, wherein x and y are each integers, means that the group may contain from x carbon atoms to y carbon atoms per group.

As used herein, the term “halo” means a halogen or halide radical and includes, for example, fluoride (F), chloride (Cl), bromide (Br), iodide (I), and astatide (At).

As used herein, the term “alkyl” means a monovalent straight, branched or cyclic saturated hydrocarbon radical, more typically, a monovalent straight or branched saturated (C₁-C₄₀)hydrocarbon radical, such as, for example, methyl, ethyl, n-propyl, isopropyl, n-butyl, isobutyl, tert-butyl, hexyl, octyl, hexadecyl, octadecyl, eicosyl, behenyl, tricontyl, and tetracontyl. As used herein, the term “cycloalkyl” means a saturated hydrocarbon radical, more typically a saturated (C₅-C₂₂) hydrocarbon radical, that includes one or more cyclic alkyl rings, which may optionally be substituted on one or more carbon atoms of the ring with one or two (C₁-C₆)alkyl groups per carbon atom, such as, for example, cyclopentyl, cycloheptyl, cyclooctyl.

As used herein, the term “alkenyl” means an unsaturated straight or branched hydrocarbon radical, more typically an unsaturated straight, branched, (C₂-C₂₂) hydrocarbon radical, that contains one or more carbon-carbon double bonds, including, for example, ethenyl (vinyl), n-propenyl, and iso-propenyl, and allyl.

As used herein, the term “alkynyl” means an unsaturated straight or branched hydrocarbon radical, more typically an unsaturated straight, branched, (C₂-C₂₂) hydrocarbon radical, that contains one or more carbon-carbon triple bonds, including, for example, ethynyl, propynyl, and butynyl.

The term “heteroalkyl” means an alkyl group wherein one or more of the carbon atoms within the alkyl group has been replaced by a hetero atom, such as, for example, nitrogen, oxygen, or sulfur.

The term “heteroalkenyl” means an alkenyl group wherein one or more of the carbon atoms within the alkenyl group has been replaced by a hetero atom, such as, for example, nitrogen, oxygen, or sulfur.

The term “heteroalkynyl” means an alkynyl group wherein one or more of the carbon atoms within the alkynyl group has been replaced by a hetero atom, such as, for example, nitrogen, oxygen, or sulfur.

As used herein, the term “aryl” means a monovalent unsaturated hydrocarbon radical containing one or more six-membered carbon rings in which the unsaturation may be represented by three conjugated double bonds. Aryl radicals include monocyclic aryl and polycyclic aryl. “Polycyclic aryl” refers to a monovalent unsaturated hydrocarbon radical containing more than one six-membered carbon ring in which the unsaturation may be represented by three conjugated double bonds wherein adjacent rings may be linked to each other by one or more bonds or divalent bridging groups or may be fused together. Aryl radicals may be substituted at one or more carbons of the ring or rings with any substituent described herein. Examples of aryl radicals include, but are not limited to, phenyl, methylphenyl, isopropylphenyl, tert-butylphenyl, methoxyphenyl, dimethylphenyl, trimethylphenyl, chlorophenyl, trichloromethylphenyl, triisobutyl phenyl, anthracenyl, naphthyl, phenanthrenyl, fluorenyl, and pyrenyl.

As used herein, the term “heterocycle” or “heterocyclic” refers to compounds having a saturated or partially unsaturated cyclic ring structure that includes one or more hetero atoms in the ring. The term “heterocyclyl” refers to a monovalent group having a saturated or partially unsaturated cyclic ring structure that includes one or more hetero atoms in the ring. Examples of heterocyclyl groups include, but are not limited to, morpholinyl, piperadinyl, piperazinyl, pyrrolinyl, pyrazolyl, and pyrrolidinyl.

As used herein, the term “heteroaryl” means a monovalent group having at least one aromatic ring that includes at least one hetero atom in the ring, which may be substituted at one or more atoms of the ring with hydroxyl, alkyl, alkoxyl, alkenyl, halo, haloalkyl, monocyclic aryl, or amino. Examples of heteroaryl groups include, but are not limited to, thienyl, pyrrolyl, pyridinyl, pyrimidinyl, pyrazinyl, triazinyl, pyridazinyl, tetrazolyl, and imidazolyl groups. The term “polycyclic heteroaryl” refers to a monovalent group having more than one aromatic ring, at least one of which includes at least one hetero atom in the ring, wherein adjacent rings may be linked to each other by one or more bonds or divalent bridging groups or may be fused together. Examples of polycyclic heteroaryl groups include, but are not limited to, indolyl and quinolinyl groups.

Any substituent described herein may optionally be further substituted at one or more carbon atoms with any substituent described herein and may be the same or different.

The compounds of the present invention have the structure represented by formula (I):

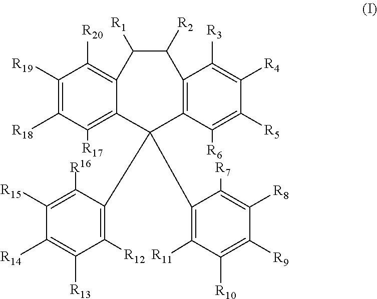

wherein
R₁, R₂, R₈, R₉, R₁₄, and R₁₅ are each, independently, a substituent selected from H, halo, cyano, hydroxyl, alkyl, alkenyl, alkynyl, heteroalkyl, heteroalkenyl, heteroalkynyl, alkoxyl,

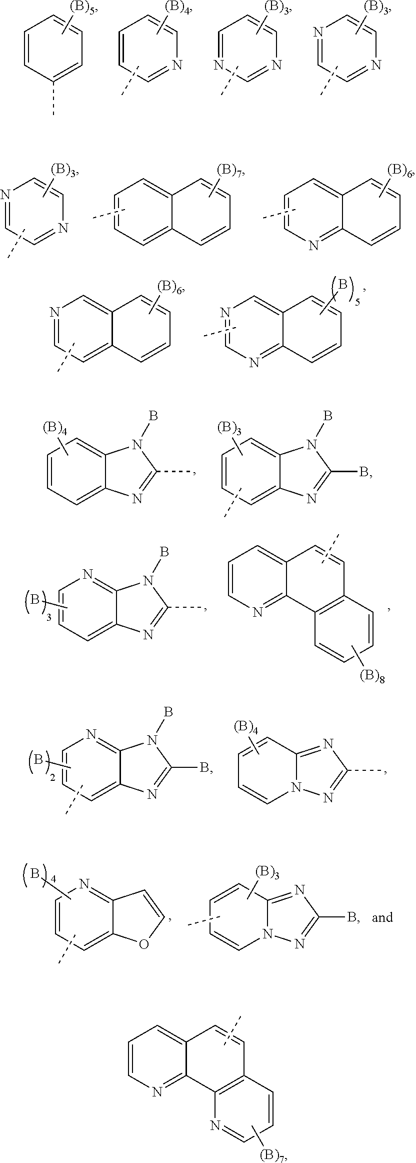

wherein each occurrence of B is, independently, a substituent selected from H, alkyl, alkenyl, alkynyl, heteroalkyl, heteroalkenyl, heteroalkynyl, aryl, heterocyclyl, and heteroaryl;
R₃, R₄, R₅, R₆, R₇, R₁₀, R₁₃, R₁₆, R₁₇, R₁₈, R₁₉, and R₂₀ are each, independently, a substituent selected from H, cyano, hydroxyl, alkyl, alkenyl, alkynyl, heteroalkyl, heteroalkenyl, heteroalkynyl, and alkoxyl;
R₁₁ and R₁₂ are each, independently, a substituent selected from H, cyano, hydroxyl, alkyl, alkenyl, alkynyl, heteroalkyl, heteroalkenyl, heteroalkynyl, and alkoxyl;
or R₁₁ and R₁₂ together form a bond;
wherein each substituent may optionally be further substituted; and
wherein at least one of R₁, R₂, R₈, R₉, R₁₄, and R₁₅ is not a substituent selected from H, cyano, hydroxyl, alkyl, alkenyl, alkynyl, heteroalkyl, heteroalkenyl, heteroalkynyl, and alkoxyl.

In an embodiment, the compound has the structure wherein
R₁₁ and R₁₂ are each, independently, a substituent selected from H, cyano, hydroxyl, alkyl, alkenyl, alkynyl, heteroalkyl, heteroalkenyl, heteroalkynyl, and alkoxyl.

In an embodiment, the compound has the structure wherein
R₁₁ and R₁₂ together form a bond.

In an embodiment, the compound has the structure wherein
R₁, R₂, R₉, and R₁₄, are each, independently, a substituent selected from H, halo,

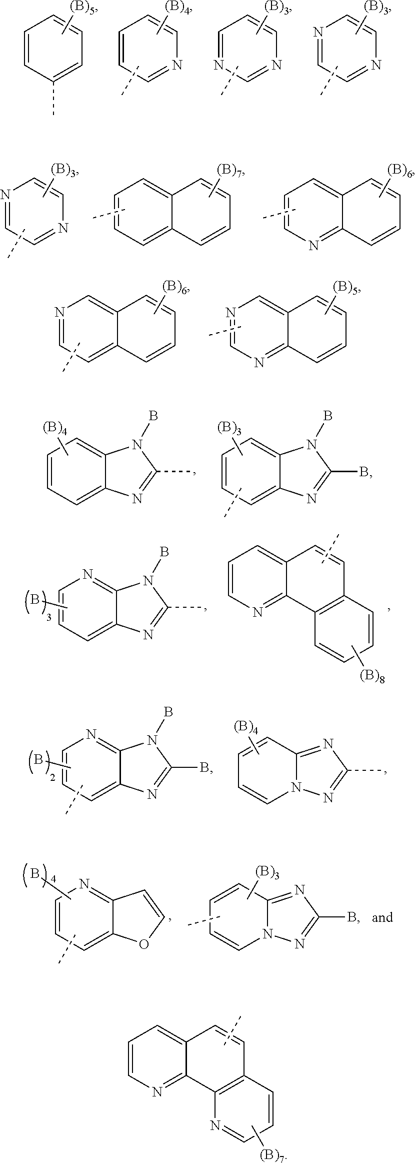

In an embodiment, the compound has the structure wherein
R₁, R₂, R₉, and R₁₄, are each, independently, a substituent selected from H, halo,

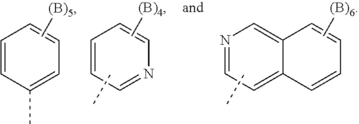

In an embodiment, the compound has the structure wherein
R₁, R₂, R₈, and R₁₅, are each, independently, a substituent selected from H, halo,

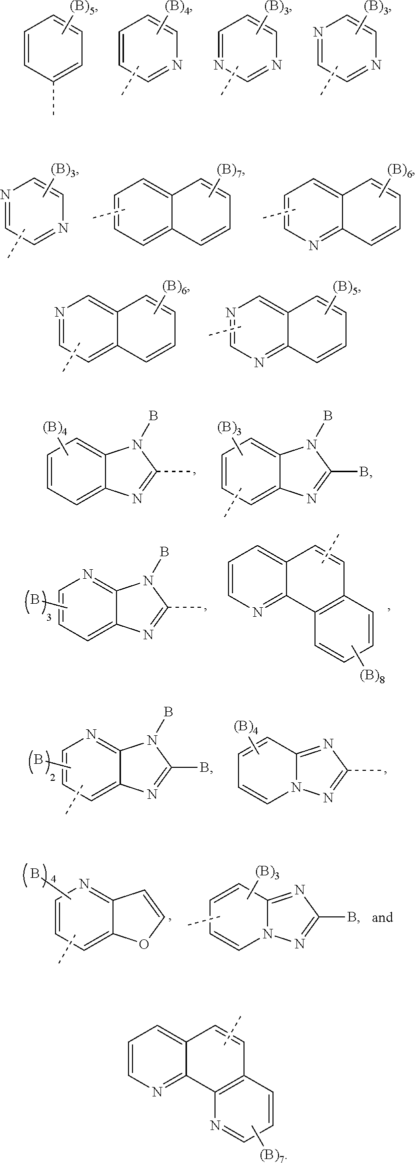

In an embodiment, the compound has the structure wherein
R₁, R₂, R₈, and R₁₅, are each, independently, a substituent selected from H, halo,

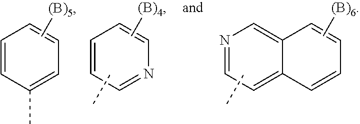

In an embodiment, the compound has the structure wherein
R₈, R₉, R₁₄, and R₁₅, are each, independently, a substituent selected from H, halo,

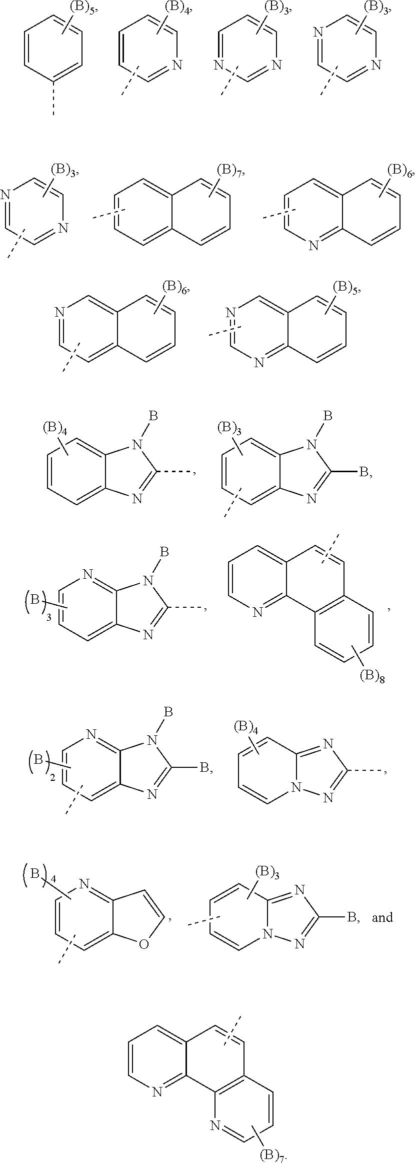

In an embodiment, the compound has the structure wherein
R₈, R₉, R₁₄, and R₁₅, are each, independently, a substituent selected from H, halo,

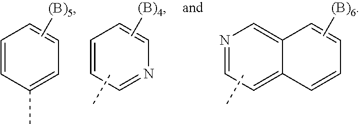

In an embodiment, the compound has the structure

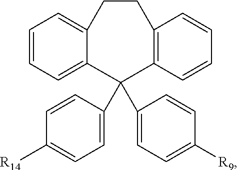

wherein R₉ and R₁₄ are each, independently, selected from H,

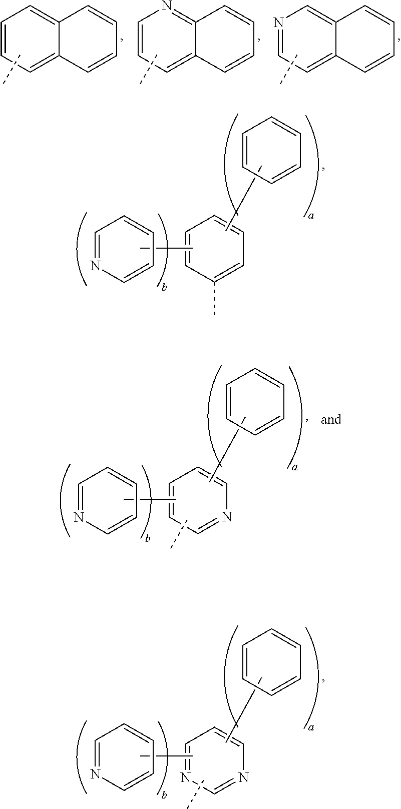

wherein a+b=0, 1, or 2.

In an embodiment, the compound has the structure

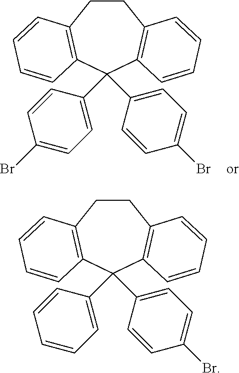 [SMILES: Brc1ccc(C2(c3ccc(Br)cc3)c3ccccc3CCc3ccccc32)cc1 ; Brc1ccc(C2(c3ccccc3)c3ccccc3CCc3ccccc32)cc1]

In an embodiment, the compound has the structure

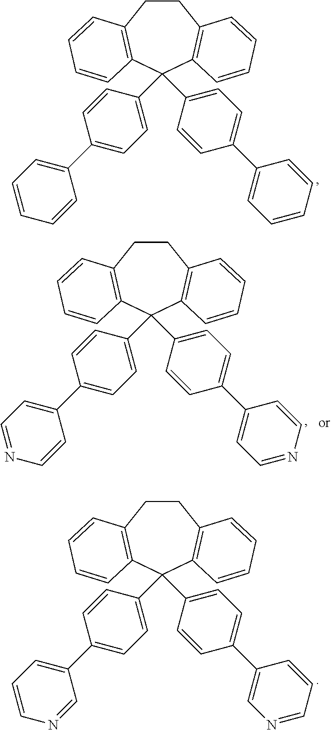

In an embodiment, the compound has the structure

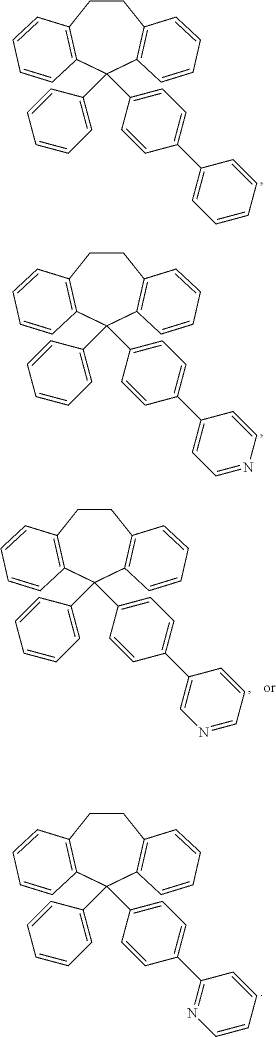

In an embodiment, the compound has the structure represented by formula (II):

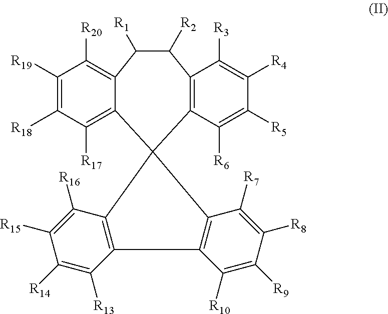

wherein
R₁, R₂, R₈, R₉, R₁₄, and R₁₅ are each, independently, a substituent selected from H, halo, cyano, hydroxyl, alkyl, alkenyl, alkynyl, heteroalkyl, heteroalkenyl, heteroalkynyl, alkoxyl,

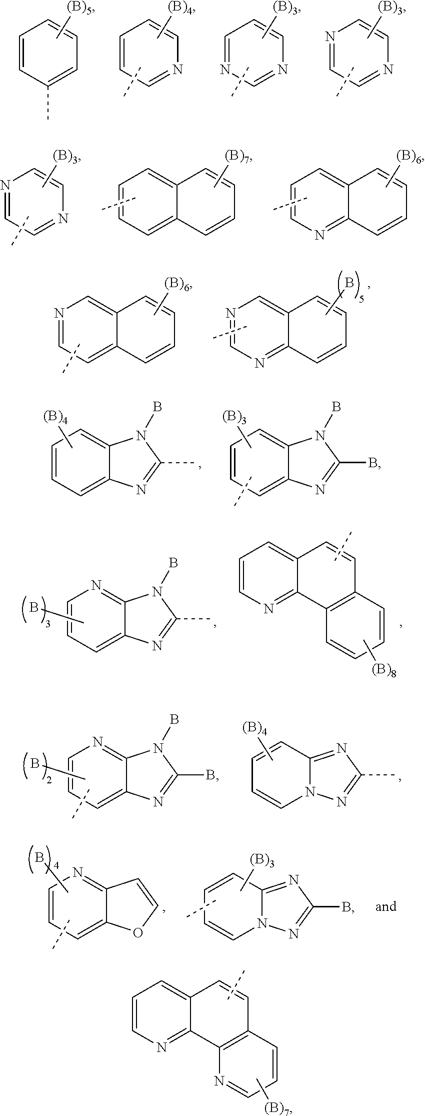

wherein each occurrence of B is, independently, a substituent selected from H, alkyl, alkenyl, alkynyl, heteroalkyl, heteroalkenyl, heteroalkynyl, aryl, heterocyclyl, and heteroaryl;
R₃, R₄, R₅, R₆, R₇, R₁₀, R₁₃, R₁₆, R₁₇, R₁₈, R₁₉, and R₂₀ are each, independently, a substituent selected from H, cyano, hydroxyl, alkyl, alkenyl, alkynyl, heteroalkyl, heteroalkenyl, heteroalkynyl, and alkoxyl;
wherein each substituent may optionally be further substituted; and
wherein at least one of R₁, R₂, R₈, R₉, R₁₄, and R₁₅ is not a substituent selected from H, cyano, hydroxyl, alkyl, alkenyl, alkynyl, heteroalkyl, heteroalkenyl, heteroalkynyl, and alkoxyl.

In an embodiment, the compound has the structure

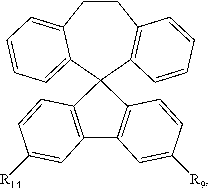

wherein R₉ and R₁₄ are each, independently, selected from H,

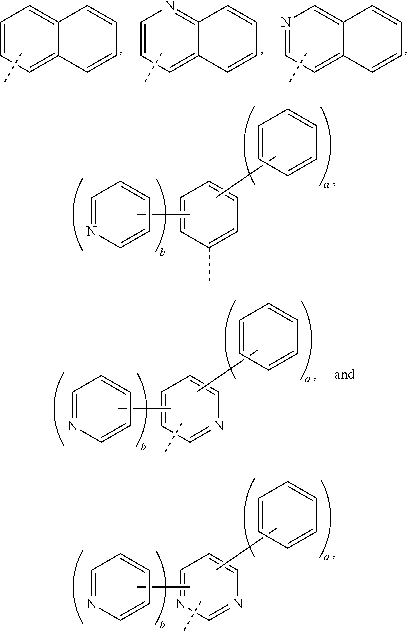

wherein a+b=0, 1, or 2.

In an embodiment, the compound has the structure

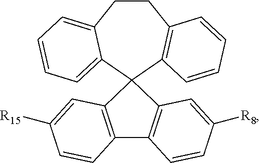

wherein R₈ and R₁₅ are each, independently, selected from H,

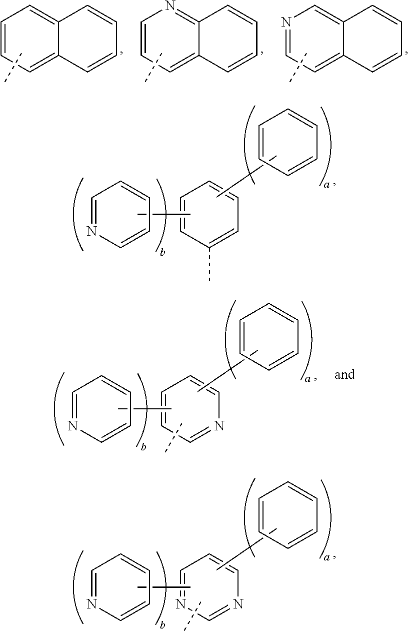

wherein a+b=0, 1, or 2.

In an embodiment, the compound has the structure

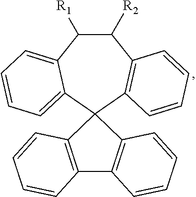

wherein R₁ and R₂ are each, independently, selected from H,

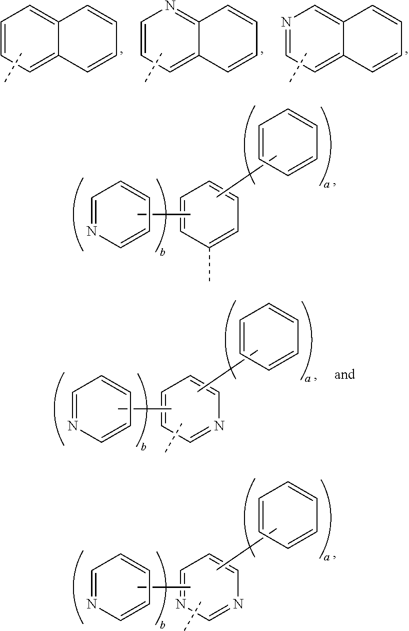

wherein a+b=0, 1, or 2.

In an embodiment, the compound has the structure

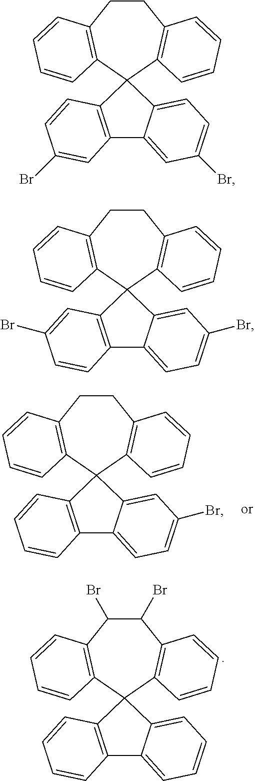

In an embodiment, the compound has the structure

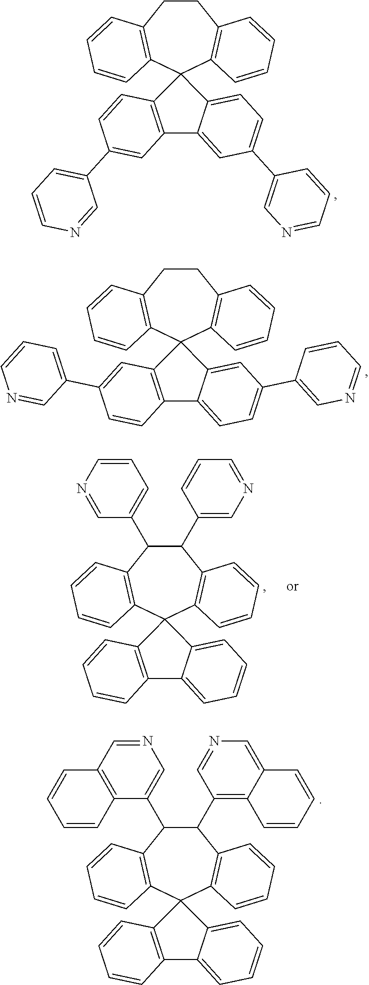

The compounds of the present invention are made according to a general process shown in Scheme 1.

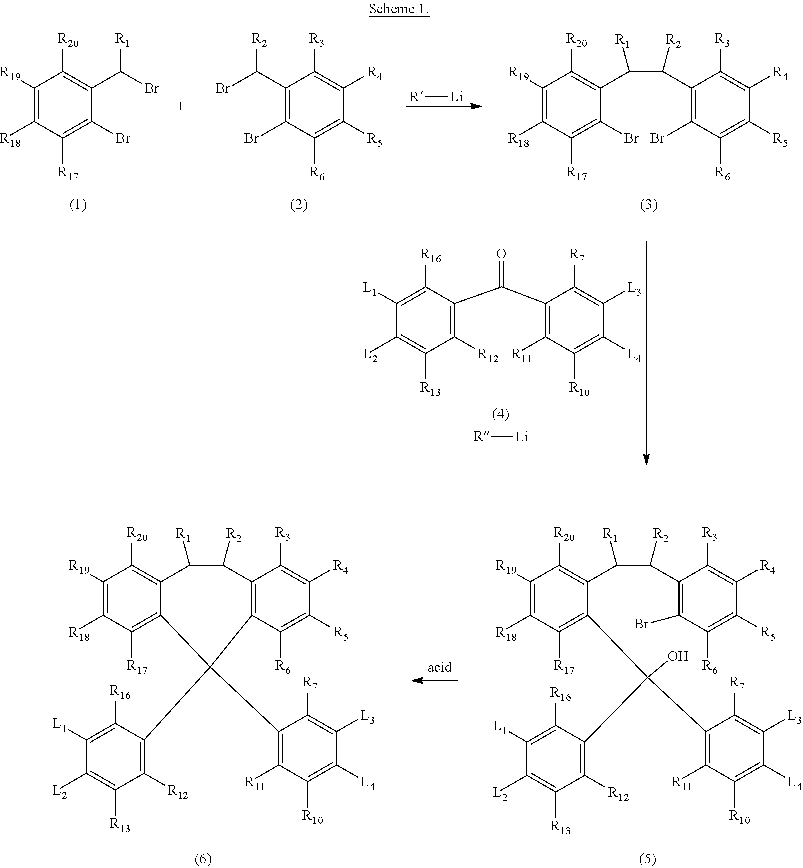

In general, compound 1 and compound 2, which can be the same or different, are reacted together in the presence of a first lithiation agent R′—Li to form compound 3. Compound 3 is then reacted with a benzophenone compound 4 in the presence of a second lithiation agent R″—Li to form compound 5, which is subsequently cyclized in the presence of an acid.

L₁, L₂, L₃, and L₄ are each, independently, a substituent selected from H, halo, trifluoromethanesulfonyl, cyano, hydroxyl, alkyl, alkenyl, alkynyl, heteroalkyl, heteroalkenyl, heteroalkynyl, and alkoxyl. R₁-R₂₀ are as defined herein.

In an embodiment, L₁, L₂, L₃, and L₄ are each, independently, H, halo, or trifluoromethanesulfonyl, and at least one of L₁, L₂, L₃, and L₄ is other than H.

R′ and R″ are the same or different, and are each, independently, alkyl. In an embodiment, R′ and R″ are each (C₁-C₅)alkyl. In another embodiment, R′ and R″ are each n-butyl.

Suitable acids include, but are not limited to, hydrogen halides, such as, for example, hydrofluoric acid (HF), hydrochloric acid (HCl), hydrobromic acid (HBr), hydroiodic acid (Hl); oxoacids, such as for example, hypochlorous acid (HClO), chlorous acid (HClO₂), chloric acid (HClO₃), perchloric acid (HClO₄), sulfuric acid (H₂SO₄), nitric acid (HNO₃), and phosphoric acid (H₃PO₄); carboxylic acids, such as, for example, acetic acid (CH₃COOH), formic acid (HCOOH), and oxalic acid (HOOC—COOH); solutions thereof and mixtures thereof.

In an embodiment, the acid comprises acetic acid, sulfuric acid, or a mixture thereof.

In an embodiment, the acid comprises acetic acid, hydrochloric acid, or a mixture thereof.

In an embodiment, when L₁, L₂, L₃, and L₄ are each, independently, H, halo, or trifluoromethanesulfonyl, and at least one of L₁, L₂, L₃, and L₄ is other than H, compound 6 may be further reacted with a compound R′″—Z in the presence of a metal catalyst according to a general process shown in Scheme 2 to form compound 7.

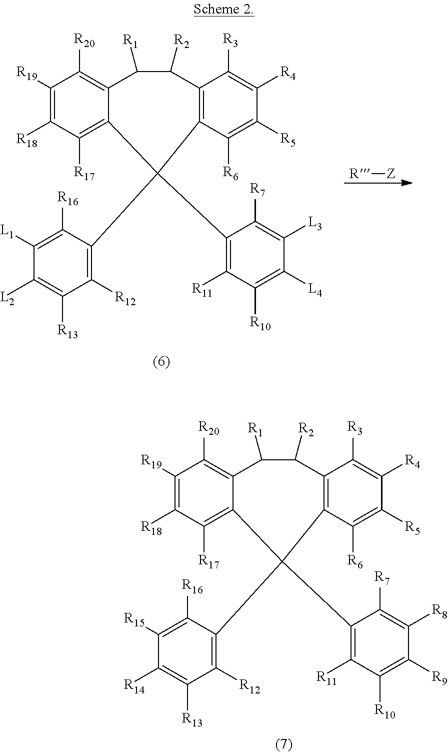

In an embodiment, R′″ is selected from

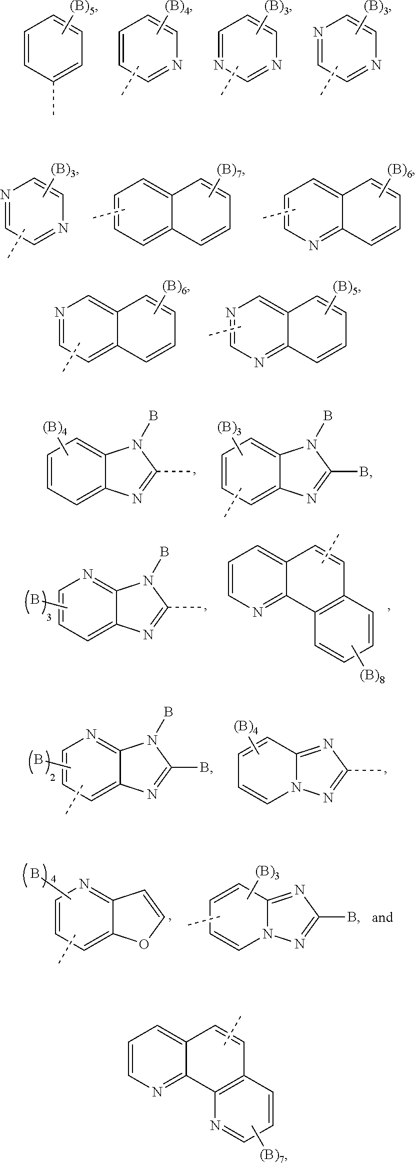

wherein each occurrence of B is, independently, a substituent selected from H, alkyl, alkenyl, alkynyl, heteroalkyl, heteroalkenyl, heteroalkynyl, aryl, heterocyclyl, and heteroaryl.

In an embodiment, Z is —B(OH)₂ or —ZnBr.

Suitable metal catalysts used in the processes of the present invention are catalysts known to those of ordinary skill in the art commonly used in Negishi cross-coupling and Suzuki cross-coupling reactions. Suitable metal catalysts include palladium catalysts, such as, for example, tetrakis(triphenylphosphine)palladium(0), bis(triphenylphosphine)palladium(II) dichloride, palladium(II) chloride, palladium(II) acetate, allylpalladium(II) chloride, bis(dibenzylideneacetone)palladium(0), bis(triphenylphosphine)palladium(II) dichloride, bis(triphenylphosphine)palladium(II) diacetate, and the like; and nickel catalysts, such as, for example, [1,3-bis(diphenylphosphino)propane]nickel(II) dichloride, bis(triphenylphosphine)nickel(II) dichloride, [1,2-Bis(diphenylphosphino)ethane]nickel(II) dichloride, [1,1′-bis(diphenylphosphino)ferrocene]nickel(II) dichloride, bis(tricyclohexylphosphine)nickel(II) dichloride, and the like.

In an embodiment, the metal catalyst is a palladium catalyst. In an embodiment, the palladium catalyst is tetrakis(triphenylphosphine)palladium(0). In an embodiment, the palladium catalyst is bis(triphenylphosphine)palladium(II) dichloride.

The compounds of the present invention may also be made according to a general process shown in Scheme 3.

According to general scheme 3, compound 8 is reacted with compound 9 in the presence of a lithiation agent R′—Li to form compound 10. Compound 10 is then cyclized by exposure to acid to form compound 11. R′ is as defined herein. L₅ and L₆ are each, independently, a substituent selected from H, halo, trifluoromethanesulfonyl, cyano, hydroxyl, alkyl, alkenyl, alkynyl, heteroalkyl, heteroalkenyl, heteroalkynyl, alkoxyl.

In an embodiment, L₅ and L₆ are each, independently, H, halo, or trifluoromethanesulfonyl, and at least one of L₅ and L₆ is other than H.

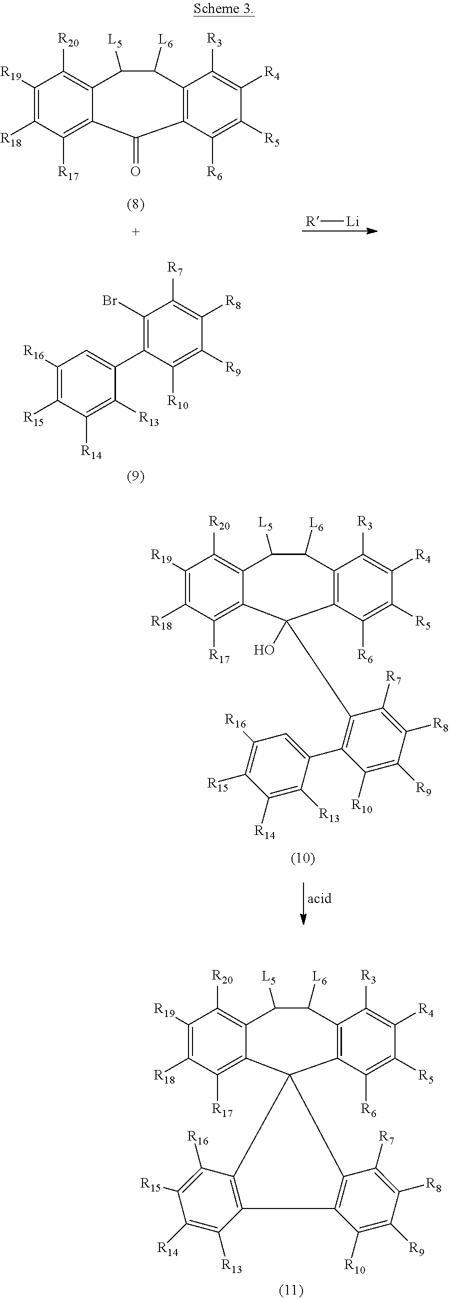

In an embodiment, when L₅ and L₆ are each, independently, H, halo, or trifluoromethanesulfonyl, and at least one of L₅ and L₆ is other than H, compound 11 may further be reacted with a compound R′″—Z to form compound 12 according to general scheme 4 in the presence of a metal catalyst defined herein. R′″ and Z are as defined herein.

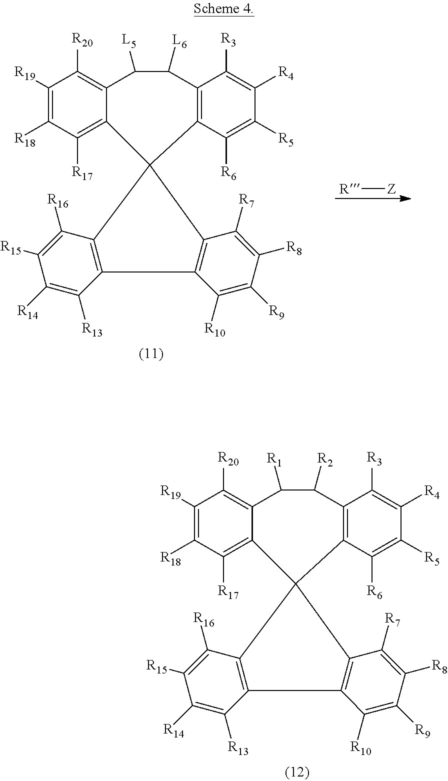

The reagents used in the processes of the present invention, such as, for example, compounds 1, 2, 4, 8, 9, lithiation agent, and acid, may be commercially-available or synthesized using synthetic methods known in the art. Suitable synthetic methods may be found in reference texts well-known in the art, such as, for example, March's Advanced Organic Chemistry, 7^{th} ed. (M. B. Smith; Wiley) and Advanced Organic Chemistry (Carey & Sundberg; Springer).

The photophysical, electrochemical, and thermal properties of the compounds of the present invention can be characterized using standard methods and apparatuses known to those of ordinary skill in the art. Ultraviolet-visible (UV-Vis) spectra may be obtained with a spectrophotometer, such as, for example, Perkin-Elmer model Lambda 900 UV/vis/near-IR spectrophotometer. Photoluminescence (PL) spectra may be obtained using a spectrofluoroimeter, such as, for example, a Photon Technology International (PTI) Inc. Model QM 2001-4 spectrofluorimeter.

UV-Vis absorption and solution PL emission spectra of the compounds of the present invention may be obtained in dilute toluene solution. Solid PL spectra may be obtained from thin films comprising the compounds of the present invention prepared by vacuum evaporation. Optical band gap (E_{g}^{opt}) may be obtained by optical transmittance measurements using known methods.

Triplet energy values (E_{T}) of the compounds of the present invention may be obtained from photoluminescence at 77K using liquid nitrogen. Differential scanning calorimeter (DSC) measurements were performed using standard methods. For example, melting point (Tₘ) and glass transition temperature (T_{g}) may be determined using a TA Instruments Q100 under nitrogen at a heating rate of 10° C./min. Thermogravimetric analysis (TGA) may be measured using standard methods, for example, by using a TA Instruments Q50 TGA instrument under nitrogen at a heating rate of 20° C./min. Energy levels may be determined via cyclic voltammetry (CV) methods. As used herein, the onset decomposition temperature (T_{d}) is the temperature at which a substance begins to decompose.

In some embodiments, the compounds of the present invention have an emission wavelength between about 150 nm to about 550 nm, typically about 200 nm to about 500 nm, more typically between about 250 nm to about 450 nm.

In some embodiments, the compounds of the present invention have triplet energy from about 2.15 eV to about 3.75 eV, typically from about 2.30 eV to about 3.60 eV, more typically from about 2.45 eV to about 3.29 eV.

In some embodiments, the compounds of the present invention have a melting temperature from about 140° C. to about 220° C., typically from about 154° C. to about 200° C.

In some embodiments, the compounds of the present invention have an onset decomposition temperature of at least 300° C. In an embodiment, the compounds of the present invention have an onset decomposition temperature from about 320° C. to about 440° C.

In some embodiments, the compounds of the present invention have an optical band gap from about 2.50 eV to about 4.50 eV, typically from about 3.00 eV to about 4.30 eV, more typically from about 3.20 eV to about 4.00 eV.

In some embodiments, the compounds of the present invention have a LUMO of about −2.80 eV to about −2.30 eV, typically about −2.71 eV to about −2.32 eV when calculated from the reduction onset potential of cyclic voltammetry curves.

In some embodiments, the compounds of the present invention have a HOMO of about −7.50 eV to about −5.00 eV, typically about −6.40 eV to about −5.50 eV, more typically about −6.33 eV to about −5.80 eV, when calculated from the reduction onset potential of cyclic voltammetry curves.

Compositions comprising at least one of the compounds of the present invention may be prepared.

In an embodiment, the composition comprises at least one compound having a structure represented by formula (I).

In an embodiment, the composition comprises at least one compound having a structure represented by formula (I) and a liquid carrier.

The liquid carrier used to form the compositions of the present invention may comprise any solvent capable of dissolving the at least one compound having a structure represented by formula (I). Typically, the liquid carrier comprises an organic solvent. The liquid carrier may be halogenated or non-halogenated and may be aromatic or non-aromatic. Suitable liquid carriers include, but are not limited to, dichloromethane, ethyl acetate, acetone, acetonitrile, dimethyl formamide, dimethyl sulfoxide, tetrahydrofuran, chlorobenzene, chloroform; (C₁-C₆)alkanols, such as methanol, ethanol, and propanol; glycols, such as ethylene glycol; and mixtures thereof.

In an embodiment, the composition of the present invention optionally further comprises a luminescent or emitter material. Suitable emitters are known in the art and can be selected to provide different emission wavelengths and colors including red, green, and blue. Emitters can be phosphorescent materials.

The weight percent of the emitter material when mixed with, for example, the compound of formula (I) can be any suitable concentration for a particular need. Typically, the composition comprises from 0% to about 40%, more typically about 1% to about 25%, even more typically about 5% to about 25% by weight of the emitter material with respect to the total weight of the composition.

Ink compositions comprising at least one of the compounds of the present invention may be prepared. In an embodiment, the ink composition comprises at least one liquid carrier and at least one compound having a structure represented by formula (I).

The compound having a structure represented by formula (I) may be used in a device, typically, an organic electronic device, or as an electron transport layer and/or hole and exciton blocking layer in an organic electronic device.

The electronic device of the present invention may be any device that comprises one or more layers of semiconductor materials and makes use of the controlled motion of electrons and holes through such one or more layers, such as, for example:
a device that converts electrical energy into radiation, such as, for example, a light-emitting diode, light emitting diode display, diode laser, or lighting panel,
a device that detects signals through electronic processes, such as, for example, a photodetector, photoconductive cell, photoresistor, photoswitch, phototransistor, phototube, infrared (“IR”) detector, or biosensor,
a device that converts radiation into electrical energy, such as, for example, a photovoltaic device or solar cell, and
a device that includes one or more electronic components with one or more semiconductor layers, such as, for example, a transistor or diode.

In an embodiment, the device comprises at least one compound having the structure represented by formula (I).

In an embodiment, the device comprises one or several layers comprising at least one compound having the structure represented by formula (I).

In an embodiment, the electronic device of the present invention comprises:
(a) an anode layer,
(b) a hole transport layer,
(c) an electroactive layer,
(d) an electron transport layer, and
(e) a cathode layer,
wherein at least one of layers (a)-(e) comprises a compound having the structure represented by formula (I).

In an embodiment, the electronic device may optionally further comprise one or more buffer layers.

In an embodiment, the electronic device may optionally further comprise one or more additional electroactive layers.

In an embodiment, the device is an organic electronic device.

In an embodiment, the device is an organic light emitting diode, an organic field-effect transistor, or an organic photovoltaic cell.

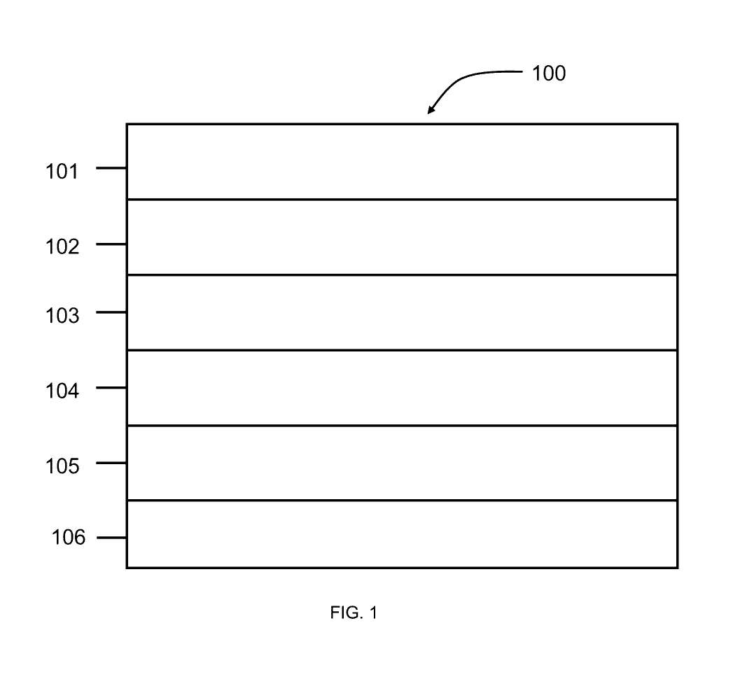

In an embodiment, the electronic device of the present invention is an electronic device 100, as shown in FIG. 1, having an anode layer 101, hole transport layer 103, an electroactive layer 104, an electron transport layer 105, wherein the electron transport layer comprises a compound having the structure represented by formula (I), and a cathode layer 106. Electronic device 100 may optionally further comprise a buffer layer 102.

The device 100 may further include a support or substrate (not shown), that can be adjacent to the anode layer 101 or the cathode layer 106. The support can be flexible or rigid, organic or inorganic. Suitable support materials include, for example, glass, ceramic, metal, and plastic films.

In one embodiment, anode layer 101 comprises mixed oxides of Groups 12, 13 and 14 elements, such as indium-tin-oxide. As used herein, the phrase “mixed oxide” refers to oxides having two or more different cations selected from the Group 2 elements or the Groups 12, 13, or 14 elements. Suitable materials used for the anode layer 101 include, but are not limited to, indium-tin-oxide (“ITO”), indium-zinc-oxide, aluminum-tin-oxide, gold, silver, copper, and nickel. The mixed oxide layer may be formed by a chemical or physical vapor deposition process or spin-cast process. Chemical vapor deposition may be performed as a plasma-enhanced chemical vapor deposition (“PECVD”) or metal organic chemical vapor deposition (“MOCVD”). Physical vapor deposition can include all forms of sputtering, including ion beam sputtering, as well as e-beam evaporation and resistance evaporation. Specific forms of physical vapor deposition include radio frequency magnetron sputtering and inductively-coupled plasma physical vapor deposition (“IMP-PVD”). These deposition techniques are well known within the semiconductor fabrication arts.

In one embodiment, the mixed oxide layer is patterned. The pattern may vary as desired. The layers can be formed in a pattern by, for example, positioning a patterned mask or resist on the first flexible composite barrier structure prior to applying the first electrical contact layer material. Alternatively, the layers can be applied as an overall layer (also called blanket deposit) and subsequently patterned using, for example, a patterned resist layer and wet chemical or dry etching techniques. Other processes for patterning that are well known in the art can also be used.

A buffer layer 102 may be absent or present depending on the intended function of the electronic device. In an embodiment, the buffer layer 102 is absent.

In some embodiments, the hole transport layer 103 is disposed between anode layer 101 and electroactive layer 104, or, in those embodiments that comprise optional buffer layer 102, between buffer layer 102 and electroactive layer 104. Hole transport layer 103 may comprise one or more hole transporting molecules and/or polymers. Commonly used hole transporting molecules include, but are not limited to: MoO₃; 4,4′,4″-tris(N,N-diphenyl-amino)-triphenylamine (TDATA), 4,4′,4″-tris(N-3-methylphenyl-N-phenyl-amino)-triphenylamine (MTDATA), N,N′-diphenyl-N,N′-bis(3-methylphenyl)-[1,1′-biphenyl]-4,4′-diamine (TPD), 1,1-bis[(di-4-tolylamino)phenyl]cyclohexane (TAPC), N,N′-bis(4-methylphenyl)-N,N′-bis(4-ethylphenyl)-[1,1′-(3,3′-dimethyl)bip-henyl]-4,4′-diamine (ETPD), tetrakis-(3-methylphenyl)-N,N,N′,N′-2,5-phenylenediamine (PDA), α-phenyl-4-N,N-diphenylaminostyrene (TPS), p-(diethylamino)benzaldehyde diphenylhydrazone (DEH), triphenylamine (TPA), bis[4-(N,N-diethylamino)-2-methylphenyl](4-methylphenyl)methane (MPMP), 1-phenyl-3-[p-(diethylamino)styryl]-5-[p-(diethylamino)phenyl]pyrazoline (PPR or DEASP), 1,2-trans-bis(9H-carbazol-9-yl)cyclobutane (DCZB), N,N,N′,N′-tetrakis(4-methylphenyl)-(1,1′-biphenyl)-4,4′-diamine (TTB), N,N′-bis(naphthalen-1-yl)-N,N′-bis-(phenyl)benzidine (α-NPB), and porphyrinic compounds, such as copper phthalocyanine. Commonly used hole transporting polymers include, but are not limited to, poly(N-vinylcarbazole) (PVK), (phenylmethyl)polysilane, poly(dioxythiophenes), such as for example, poly(3,4-ethylenedioxythiophene):poly(styrenesulfonate) (PEDOT:PSS), polyanilines, and polypyrroles. It is also possible to obtain hole transporting polymers by doping hole transporting molecules, such as those mentioned above, into polymers such as polystyrene, polycarbonate, and poly(3,4-ethylenedioxythiophene):poly(styrenesulfonate).

Electron transport layer 105 comprises a compound having the structure represented by formula (I).

In an embodiment, electron transport layer 105 optionally further comprises additional electron transport materials. Examples of additional electron transport materials include, for example, metal chelated oxinoid compounds, such as bis(2-methyl-8-quinolinolato)(para-phenyl-phenolato)aluminum(III) (BAIQ) and tris(8-hydroxyquinolato)aluminum, tetrakis(8-hydroxyquinolinato)zirconium, azole compounds such as 2-(4-biphenylyl)-5-(4-t-butylphenyl)-1,3,4-oxadiazole (PBD), 3-(4-biphenylyl)-4-phenyl-5-(4-t-butylphenyl)-1,2,4-triazole (TAZ), and 1,3,5-tri(phenyl-2-benzimidazole)benzene (TPBI), quinoxaline derivatives such as 2,3-bis(4-fluorophenyl)quinoxaline, phenanthroline derivatives such as 9,10-diphenylphenanthroline (DPA) and 2,9-dimethyl-4,7-diphenyl-1,10-phenanthroline (DDPA), and 1,3-bis(5-(4-tert-butylphenyl)-1,3,4-oxadiazol-2-yl)benzene (OXD-7), as well as mixtures thereof. Alternatively, the electron transport layer 105 may optionally further comprise an inorganic material, such as, for example, BaO, LiF, Li₂O.

The composition of electroactive layer 104 depends on the intended function of device 100, for example, electroactive layer 104 can be a light-emitting layer (emissive layer) that is activated by an applied voltage (such as in a light-emitting diode or light-emitting electrochemical cell), or a layer of material that responds to radiant energy and generates a signal with or without an applied bias voltage (such as in a photodetector).

In an embodiment, electroactive layer 104 is an emissive layer.

In an embodiment, electroactive layer 104 comprises an organic electroluminescent (“EL”) material, or emitter material, such as, for example, electroluminescent small molecule organic compounds, electroluminescent metal complexes, and electroluminescent conjugated polymers, as well as mixtures thereof. Suitable EL small molecule organic compounds include, for example, pyrene, perylene, rubrene, and coumarin, as well as derivatives thereof and mixtures thereof. Suitable EL metal complexes include, for example, metal chelated oxinoid compounds, such as tris(8-hydroxyquinolate)aluminum, cyclo-metallated iridium and platinum electroluminescent compounds, such as complexes of iridium with phenylpyridine, phenylquinoline, or phenylpyrimidine ligands as disclosed in Petrov et al., U.S. Pat. No. 6,670,645, and organometallic complexes such as those described in, for example, Published PCT Applications WO 03/008424, WO 03/091688, and WO 03/040257, as well as mixtures any of such EL metal complexes. Examples of suitable EL metal complexes include, but are not limited to, tris(5-phenyl-10,10-dimethyl-4-aza-tricycloundeca-2,4,6-triene)iridium(III) [Ir(pppy)₃], tris(2-phenylpyridine)iridium(III) [Ir(ppy)₃] and bis(3,5-difluoro-2-(2-pyridyl)phenyl-(2-carboxypyridyl)iridium (III) [FIr(pic)].

The organic electroluminescent material or emitter material of electroactive layer 104 may be chosen according to the color of light desired. In an embodiment, electroactive layer 104 comprises a blue emitter, a green emitter, a red emitter, or a combination thereof.

In an embodiment, the electroactive layer 104 optionally further comprises hole transporting molecules and/or polymers, electron transport materials, or a combination thereof.

Materials suitable for use as cathode layer 106 are known in the art and include, for example, alkali metals of Group 1, such as Li, Na, K, Rb, and Cs, Group 2 metals, such as, Mg, Ca, Ba, Group 12 metals, lanthanides such as Ce, Sm, and Eu, and actinides, as well as aluminum, indium, yttrium, and combinations of any such materials. Specific non-limiting examples of materials suitable for cathode layer 106 include, but are not limited to, Barium, Lithium, Cerium, Cesium, Europium, Rubidium, Yttrium, Magnesium, Samarium, and alloys and combinations thereof. Cathode layer 106 is typically formed by a chemical or physical vapor deposition process. In some embodiments, the cathode layer may be patterned, as described herein with reference to the anode layer 101.

Though not shown in FIG. 1, it is understood that device 100 may comprise additional layers. Other layers that are known in the art or otherwise may be used. In addition, any of the above-described layers may comprise two or more sub-layers or may form a laminar structure. Alternatively, some or all of anode layer 101, buffer layer 102, hole transport layer 103, electron transport layer 105, cathode layer 106, and any additional layers may be treated, especially surface treated, to increase charge carrier transport efficiency or other physical properties of the devices. The choice of materials for each of the component layers is typically determined by balancing the goals of providing a device with high device efficiency with device operational lifetime considerations, fabrication time and complexity factors and other considerations appreciated by persons skilled in the art. It will be appreciated that determining optimal components, component configurations, and compositional identities would be routine to those of ordinary skill of in the art.

The various layers of the electronic device can be formed by any conventional deposition technique, including vapor deposition, liquid deposition (continuous and discontinuous techniques), and thermal transfer. Continuous deposition techniques, include but are not limited to, spin coating, gravure coating, curtain coating, dip coating, slot-die coating, spray coating, roll-to-roll coating, and continuous nozzle coating. Discontinuous deposition techniques include, but are not limited to, ink jet printing, gravure printing, and screen printing. Other layers in the device can be made of any materials which are known to be useful in such layers upon consideration of the function to be served by such layers.

As is known in the art, the location of the electron-hole recombination zone in the device, and thus the emission spectrum of the device, can be affected by the relative thickness of each layer. The appropriate ratio of layer thicknesses will depend on the exact nature of the device and the materials used. Typically, the thickness of the anode layer, the cathode layer, the electroactive layer, the hole transport layer, the electron transport layer, and optional buffer layer, when present, are each from about 0.001-1000 μm, more typically about 0.005-100 μm, even more typically about 0.01-10 μm, yet even more typically about 0.02-1 μm.

In one embodiment, the electronic device of the present invention is a device for converting electrical energy into radiation, and comprises an anode 101, a cathode layer 106, an electroactive layer 104 that is capable of converting electrical energy into radiation, disposed between the anode layer 101 layer and the cathode layer 106, a hole transport layer 103, an electron transport layer 105 comprising a compound represented by formula (I), and optionally further comprising a buffer layer 102. In one embodiment, the device is a light emitting diode (“LED”) device and the electroactive layer 104 of the device is an electroluminescent material, even more typically, and the device is an organic light emitting diode (“OLED”) device and the electroactive layer 104 of the device is organic electroluminescent material. In one embodiment, the OLED device is an “active matrix” OLED display, wherein, individual deposits of photoactive organic films may be independently excited by the passage of current, leading to individual pixels of light emission. In another embodiment, the OLED is a “passive matrix” OLED display, wherein deposits of photoactive organic films may be excited by rows and columns of electrical contact layers.

Characteristics of the electronic device of the present invention may be determined using standard methods and apparatuses known in the art. For example, film thickness may be measured using a profilometer. Electroluminescence (EL) spectra may be obtained using a spectrofluorimeter as described herein. Device performance of the devices may be measured, for example, by using a HP4155A semiconductor parameter analyzer (Yokogawa Hewlett-Packard, Tokyo). Luminance may be measured by using an optometer. Device external quantum efficiency (EQE) may be calculated from the luminance, current density and EL spectrum assuming a Lambertian distribution using known procedures.

In some embodiments, the electronic devices described herein have a turn-on voltage at a brightness of 1 cd/m² of at most about 5 V, typically of at most about 6 V, more typically of at most about 7 V.

In some embodiments, the devices described herein have a luminous (current) efficiency of at least about 20 cd/A, typically at least about 25 cd/A, more typically at least about 30 cd/A.

In some embodiments, the devices described herein have a maximum luminance that can be at least about 3500 cd/m², typically at least about 4000 cd/m², more typically at least about 5000 cd/m², even more typically at least about 7400 cd/m². In some embodiments, the devices described herein have a power efficiency of at least about 1.5 lm/W, typically of at least about 2 lm/W, more typically of at least about 3 lm/W.

In some embodiments, the devices described herein have an external quantum efficiency of at least about 4%, typically of at least about 5%, more typically of at least about 6%, even more typically of at least about 7%.

The present invention is further illustrated by the following non-limiting examples.

### Examples

GENERAL TECHNIQUES. ¹H NMR spectra were recorded on a Bruker AV300 at 300 MHz, and ¹³C NMR spectra were recorded on a Bruker AV500 at 500 MHz using CDCl₃ as the solvent. High resolution mass spectra were recorded using a JEOL/HX-110 spectrometer in FAB mode. Ultraviolet-Visible (UV-Vis) spectra were obtained with a Perkin-Elmer model Lambda 900 UV/vis/near-IR spectrophotometer and photoluminescence (PL) spectra were recorded on a Photon Technology International (PTI) Inc. Model QM 2001-4 spectrofluorimeter. UV-Vis absorption and solution PL emission spectra of the compounds were obtained from dilute toluene solution, and solid PL spectra were obtained from a thin film prepared by vacuum evaporation.

Triplet energy values of the compounds of the present invention were obtained from photoluminescence at 77K using liquid nitrogen. Differential scanning calorimeter (DSC) measurements were performed on a TA Instruments Q100 under nitrogen at a heating rate of 10° C./min to measure melting point (Tₘ) and glass transition temperature (T_{g}). Thermogravimetric analysis (TGA) was measured by TA Instruments Q50 under nitrogen at a heating rate of 20° C./min. Energy levels were measured via cyclic voltammetry (CV). Compounds were dissolved in anhydrous acetonitrile with 0.1 M tetrabutylammonium hexafluorophosphate as the electrolyte to measure the LUMO energy level. Platinum wire working and counter electrodes and a saturated Ag/AgCl reference electrode were used. Ferrocene was used as the standard material. All solutions were purged with nitrogen for 10 minutes before each experiment.

### Example 1

### Preparation of 10,11-di-3-pyridinyl-spiro[(10,11-dihydro-5H-dibenzo[a,d]cycloheptene-5,9′-fluorene)] (10,11-DPSDF)

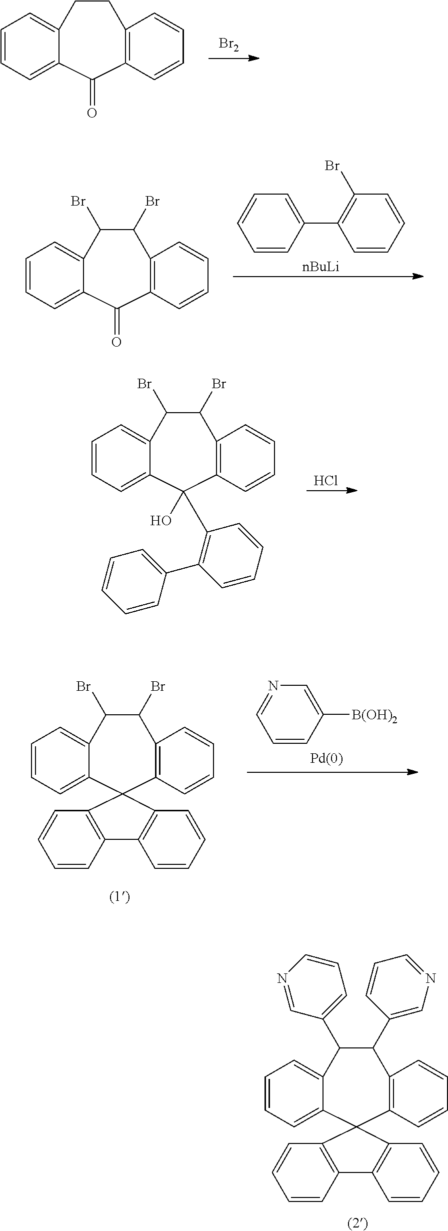

To 9-dibenzosuberone (3.0 g, 14.4 mmol) was added bromine (6.9 g, 43.2 mmol) in dichloro-methane at 0° C. under nitrogen atmosphere. After being stirred for 4 h, water and dichloromethane were added. The organic phase was separated, washed with brine solution, dried over anhydrous MgSO₄, filtered and dried to remove the solvents. Purification by recrystallization with ethanol gave 10,11-dibromo[(10,11-dihydro-5H-dibenzo[a,d]cycloheptone)] as a white solid. Yield 88%. 1H NMR (CDCl3, 300 MHz) δ 8.13-8.11 (d, 2H), 7.62-7.50 (m, 4H), 7.45-7.43 (d, 2H), 5.82 (s, 2H).

To a 250 mL two-necked flask was placed a solution of 2-bromobiphenyl (1.0 g, 4.29 mmol) in THF (20 mL). The reaction flask was cooled to −78° C. and n-butyllithium (2.5 M in n-hexane, 2.23 mL) was added dropwise slowly. The whole solution was stirred at this temperature for 2 h, followed by the addition of a solution of 10,11-dibromo[(10,11-dihydro-5H-dibenzo[a,d]cycloheptone)] (2.04 g, 5.57 mmol) in THF (40 mL) under an argon atmosphere. The resulting mixture was gradually warmed to ambient temperature and quenched by adding saturated, aqueous NaHCO₃ (100 mL). The mixture was extracted with dichloromethane. The combined organic layers were dried over MgSO₄, filtered and evaporated under reduced pressure yielding yellow powdery product. The crude residue was placed in another two-necked flask and dissolved in acetic acid (50 mL). A catalytic amount of aqueous HCl (5 mol %, 12 N) was then added and the whole solution was refluxed for 12 h. After cooling to ambient temperature, purification by silica gel chromatography using ethyl acetate/n-hexane as an eluent gave 10,11-dibromo-spiro[(10,11-dihydro-5H-dibenzo[a,d]cycloheptene-5,9′-fluorene)] (1′) as a white powder. Yield 70%. 1H NMR (CDCl3, 300 MHz) δ 7.97-7.94 (d, 2H), 7.74-7.71 (d, 2H), 7.39-7.33 (m, 2H), 7.25-7.15 (m, 6H), 6.95-6.86 (m, 6H), 5.82 (s, 2H).

A mixture of 10,11-dibromo-spiro[(10,11-dihydro-5H-dibenzo[a,d]cycloheptene-5,9′-fluorene)] (1′) (2.5 g, 4.97 mmol), 3-pyridinylboronic acid (1.68 g, 13.6 mmol) and tetrakis(triphenylphosphine)palladium(0) (5 mol %) in 20 mL of tetrahydrofuran was refluxed under argon for 12 h. To the reaction mixture was added a solution of potassium carbonate (2 M, 20 mL) dropwise slowly. After being cooled to ambient temperature, the reaction mixture was extracted with dichloromethane and water. The organic layer was evaporated with a rotary evaporator. The product was purified by column chromatography using ethyl acetate and n-hexane mixture (90:10) and 10,11-di-3-pyridinyl-spiro[(10,11-dihydro-5H-dibenzo[a,d]cycloheptene-5,9′-fluorene)] (10,11-DPSDF) (2′) as a white solid product was obtained. ¹H NMR (300 MHz, CDCl₃, ppm): δ 8.85 (s, 2H), 8.64 (s, 2H), 7.68-7.62 (m, 8H), 7.53-7.45 (m, 10H), 7.24 (m, 2H), 5.01 (s, 2H).

### Example 2

### Preparation of 10,11-Di-3-quinolinyl-spiro[(10,11-dihydro-5h-dibenzo[a,d]cycloheptene-5,9′-fluorene)] (10,11-DQSDF)

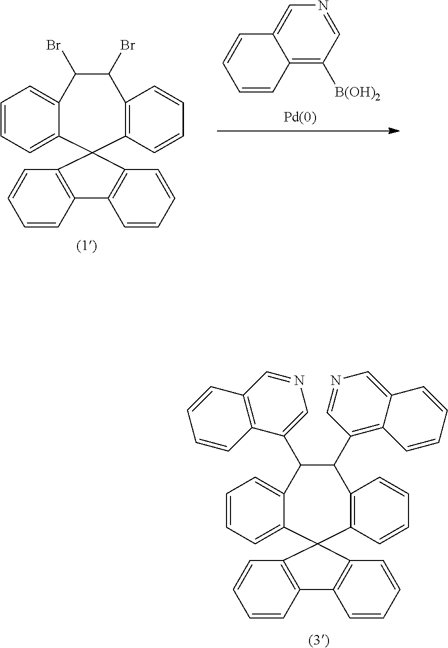 [SMILES: BrC1c2ccccc2C2(c3ccccc3-c3ccccc32)c2ccccc2C1Br ; OB(O)c1cncc2ccccc12 ; c1ccc2c(c1)-c1ccccc1C21c2ccccc2C(c2cncc3ccccc23)C(c2cncc3ccccc23)c2ccccc21]

A mixture of 10,11-dibromo-spiro[(10,11-dihydro-5H-dibenzo[a,d]cycloheptene-5,9′-fluorene)] (1′) (1.0 g, 1.99 mmol), quinoline-3-boronic acid (0.75 g, 4.38 mmol) and tetrakis(triphenylphosphine)palladium(0) (5 mol %) in 20 mL of tetrahydrofuran was refluxed under argon for 12 h. To the reaction mixture was added a solution of potassium carbonate (2 M, 20 mL) dropwise slowly. After being cooled to ambient temperature, the reaction mixture was extracted with dichloromethane and water. The organic layer was evaporated with a rotary evaporator. The product was purified by column chromatography using ethyl acetate and n-hexane mixture (90:10) and a white solid product was obtained. ¹H NMR (300 MHz, CDCl₃, ppm): δ 9.3 (s, 2H), 8.47 (s, 2H), 8.20-8.17 (d, 2H), 7.97-7.94 (m, 10H), 7.81-7.76 (m, 10H), 7.66-7.61 (d, 2H), 5.3 (s, 2H).

### Example 3

### Preparation of 2-Bromo-spiro[fluorene-9,5′-dibenzosuberane]

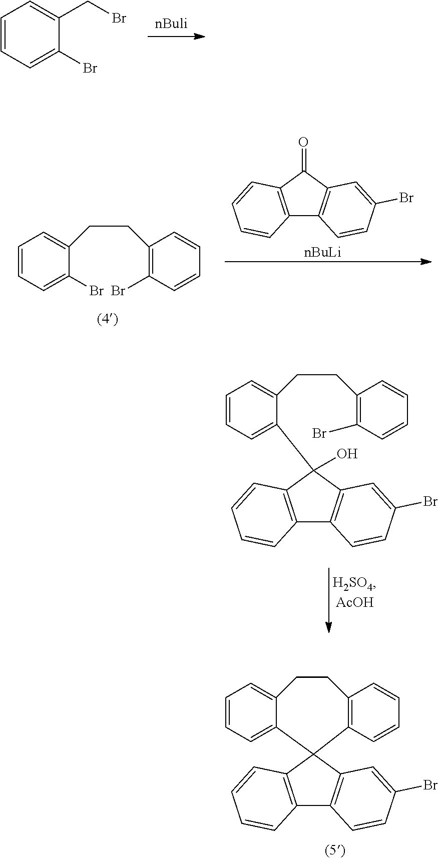

A 250 mL two-necked flask was placed a solution of 2-bromo benzylbromide (20 g, 80.0 mmol) in THF (100 mL). The reaction flask was cooled to −78° C. and n-butyllithium (2.5 M in n-hexane, 16.7 mL) was added dropwise to the stirred solution. After that, the resulting mixture was gradually warmed to ambient temperature overnight and quenched by water (100 mL). The mixture was extracted with ethyl acetate. The combined organic layers were dried over MgSO₄, filtered and evaporated under reduced pressure and recrystallized by petroleum ether to give 1,2-bis(2-bromophenyl)ethane (4′) as a white crystalline product. Yield 91.1%. ¹H NMR (CDCl₃, 300 MHz) δ 7.55 (d, 2H), 7.26-7.16 (m, 4H), 7.10-7.04 (m, 2H), 3.04 (s, 4H); GC-MS(FAB) 340 ([M+H⁺]).

To a 250 mL two-necked flask was placed a solution of 1,2-bis(2-bromophenyl)ethane (4′) (3.0 g, 8.8 mmol) in THF (30 mL). The reaction flask was cooled to −78° C. and n-butyllithium (2.5 M in n-hexane, 4.23 mL) was added dropwise slowly. The whole solution was stirred at this temperature for 2 h, followed by the addition of a solution of 2-bromo-9-fluorenone (2.7 g, 10.5 mmol) in THF (40 mL) under an argon atmosphere. The resulting mixture was gradually warmed to ambient temperature and quenched by adding saturated, aqueous NaHCO₃ (100 mL). The mixture was extracted with dichloromethane. The combined organic layers were dried over MgSO₄, filtered and evaporated under reduced pressure yielding yellow powdery product. The crude residue was placed in another two-necked flask and dissolved in acetic acid (50 mL). A catalytic amount of aqueous H₂SO₄ (10 mol %) was then added and the whole solution was refluxed for 12 h. After cooling to ambient temperature, purification by silica gel chromatography using n-hexane as an eluent gave the product 5′ as a yellow powder.

### Example 4

### Preparation of 2,7-Dibromo-spiro[fluorene-9,5′-dibenzosuberane]

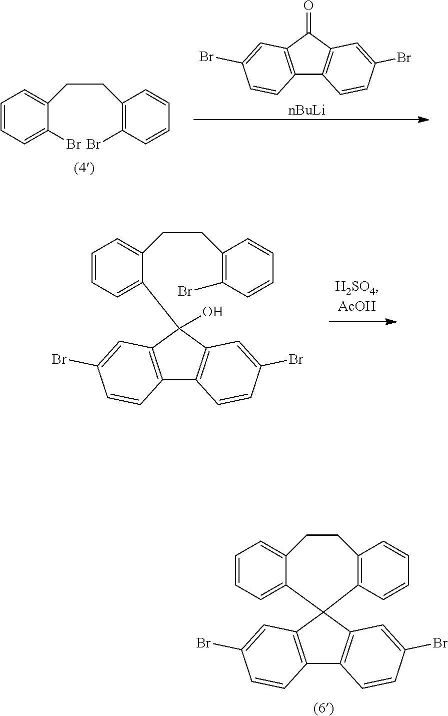

To a 250 mL two-necked flask was placed a solution of 1,2-bis(2-bromophenyl)ethane (4′) (6.0 g, 17.6 mmol) in THF (70 mL). The reaction flask was cooled to −78° C. and n-butyllithium (2.5 M in n-hexane, 9.2 mL) was added dropwise slowly. The whole solution was stirred at this low temperature for 2 h, followed by the addition of a solution of 2,7-dibromo-9-fluorenone (7.8 g, 22.9 mmol) in THF (80 mL) under an argon atmosphere. The resulting mixture was gradually warmed to ambient temperature and quenched by adding saturated, aqueous NaHCO₃ (100 mL). The mixture was extracted with dichloromethane. The combined organic layers were dried over MgSO₄, filtered and evaporated under reduced pressure yielding a yellow powdery product. The crude residue was placed in another two-necked flask and dissolved in acetic acid (100 mL). A catalytic amount of aqueous H₂SO₄ (10 mol %) was then added and the whole solution was refluxed for 12 h. After cooling to ambient temperature, purification by silica gel chromatography using n-hexane as an eluent gave a white powder. Yield 5.9 g, 67%. ¹H NMR (300 MHz, CDCl₃, ppm): δ 7.63-7.38 (m, 6H), 7.29-7.06 (m, 8H), 3.02-2.87 (m, 4H). ¹³C NMR (500 MHz, CDCl₃, ppm): δ 140.9, 132.8, 132.8, 130.6, 130.5, 128.5, 128.4, 127.8, 127.7, 127.4, 126.0, 124.5, 38.4, 36.4, 36.2; MALDI/TOF-MS 503 ([M+H]⁺).

### Example 5

### Preparation of 5,5-Bis(4-bromophenyl)-9H-dibenzosuberane

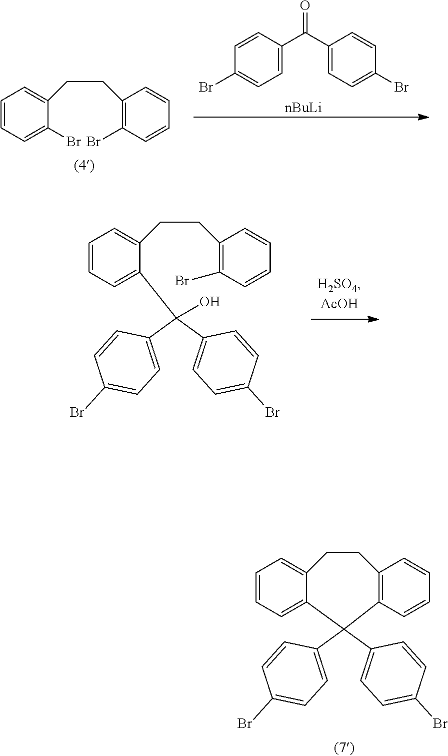

To a 250 mL two-necked flask was placed a solution of 1,2-bis(2-bromophenyl)ethane (4′) (5.8 g, 16.9 mmol) in THF (60 mL). The reaction flask was cooled to −78° C. and n-butyllithium (2.5 M in n-hexane, 8.8 mL) was added dropwise slowly. The whole solution was stirred at this temperature for 2 h, followed by the addition of a solution of 4,4′-dibromobenzophenone (7.5 g, 21.9 mmol) in THF (80 mL) under an argon atmosphere. The resulting mixture was gradually warmed to ambient temperature and quenched by adding saturated, aqueous NaHCO₃ (100 mL). The mixture was extracted with dichloromethane. The combined organic layers were dried over MgSO₄, filtered and evaporated under reduced pressure yielding yellow powdery product. The crude residue was placed in another two-necked flask and dissolved in acetic acid (100 mL). A catalytic amount of aqueous H₂SO₄ (10 mol %) was then added and the whole solution was refluxed for 12 h. After cooling to ambient temperature, purification by silica gel chromatography using n-hexane as an eluent gave 5,5-bis(4-bromophenyl)-9H-dibenzosuberane (7′) as a white powder. Yield 5.38 g, 67%. ¹H NMR (300 MHz, CDCl₃, ppm): δ 7.66-6.78 (m, 14H), 5.79-5.75 (m, 2H), 3.02-2.86 (m, 4H). ¹³C NMR (500 MHz, CDCl₃, ppm): δ 141.9, 140.3, 137.1, 136.5, 133.1, 131.7, 131.3, 130.3, 129.7, 128.4, 128.1, 127.6, 127.3, 127.0, 126.8, 124.1, 120.7, 52.4, 38.4, 36.2; MALDI/TOF-MS 505 ([M+H]⁺).

### Example 6

### Preparation of 5,5-(4-Bromophenyl)(phenyl)-9H-dibenzosuberane

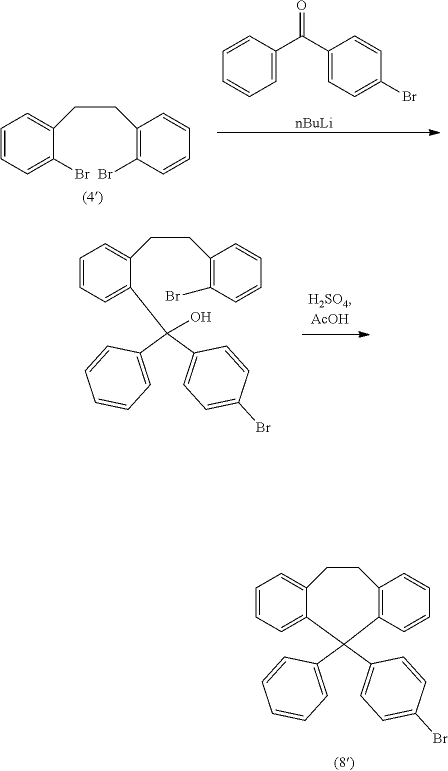

To a 250 mL two-necked flask was placed a solution of 1,2-bis(2-bromophenyl)ethane (4′) (5.0 g, 14.7 mmol) in THF (60 mL). The reaction flask was cooled to −78° C. and n-butyllithium (2.5 M in n-hexane, 7.6 mL) was added dropwise to the stirred solution. The whole solution was stirred at −78° C. for 2 h, followed by the addition of a solution of 4-bromobenzophenone (4.9 g, 19.1 mmol) in THF (10 mL) under an argon atmosphere. After that, the resulting mixture was gradually warmed to ambient temperature overnight and quenched by aqueous NaHCO₃ (5%, 100 mL). The mixture was extracted with ethyl acetate. The combined organic layers were dried over MgSO₄, filtered and evaporated under reduced pressure and vacuum dried to get a yellow powder. The crude powder was placed in another two-necked flask and dissolved in acetic acid (100 mL). A catalytic amount of H₂SO₄ (10 mL) was added and the whole solution was refluxed for 12 h. After cooling to ambient temperature, purification by silica gel chromatography using n-hexane as an eluent gave 5,5-(4-bromophenyl)(phenyl)-9H-dibenzosuberane (8′) as a white powder. Yield 3.69 g, 58%. ¹H NMR (300 MHz, CDCl₃, ppm): δ 7.67-6.83 (m, 13H), 6.63-6.61 (d, 2H), 6.09-6.07 (d, 2H), 5.38-5.32 (m, 2H), 5.17-5.14 (m, 2H). ¹³C NMR (500 MHz, CDCl₃, ppm): δ 144. 4, 141.6, 141.2, 138.2, 137.3, 133.2, 132.6, 132.3, 131.6, 131.2, 129.3, 127.3, 126.6, 123.8, 120.0, 57.9, 46.7, 36.1; GC/MS-El 425 ([M+H]⁺).

### Example 7

### Preparation of 5,5-Phenyl(4-(pyridine-2-yl)phenyl)-9H-dibenzosuberane (2PySDP)

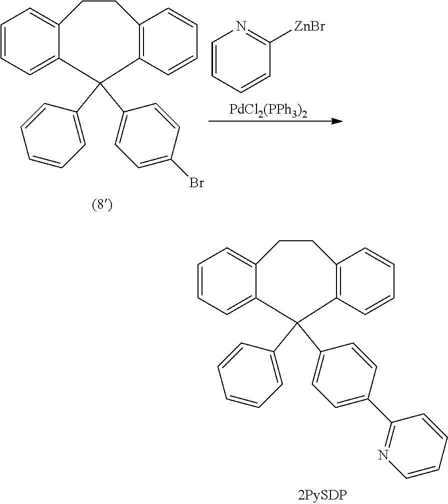

A mixture of 5,5-(4-bromophenyl)(phenyl)-9H-dibenzosuberane (8′) (2.00 g, 4.70 mmol), 2-pyridylzinc bromide (0.5 M in THF, 12.17 mL, 6.11 mmol), and PdCl₂(PPh₃)₂ (0.06 g, 0.94 mmol) in anhydrous THF (120 mL) was stirred under reflux for 24 h under an argon atmosphere. After cooling to room temperature, the mixture was poured into water and then extracted with chloroform. The combined organic phase was washed with brine and dried over MgSO₄. The crude mixture was subjected to silica gel chromatography by ethyl acetate: n-hexane mixture (1:9) which afforded 2PySDP (0.1 g, 5.2%) as white powder. Yield 5.2%. ¹H NMR (300 MHz, CDCl₃, ppm): δ 8.68 (s, 1H), 7.88-7.69 (m, 3H) 7.54-6.51 (m, 17H), 6.00-5.94 (m, 1H) 5.44-5.24 (m, 2H), 5.10-4.96 (m, 2H). ¹³C NMR (500 MHz, CDCl₃, ppm): δ 157.2, 149.6, 146.2, 144.6, 141.7, 138.5, 136.7, 133.1, 132.6, 132.2, 131.4, 131.0, 130.4, 129.0, 127.9, 127.4, 127.0, 126.6, 126.5, 126.3, 124.0, 121.9, 120.4, 58.2, 46.7, 36.0; MALDI/TOF-MS 423 ([M+H]⁺).

### Example 8

### Preparation of 5,5-Phenyl(4-(pyridine-3-yl)phenyl)-9H-dibenzosuberane (3PySDP)

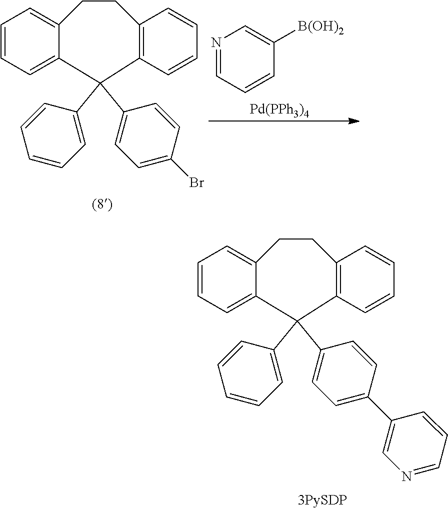

A mixture of 5,5-(4-bromophenyl)(phenyl)-9H-dibenzosuberane (8′) (2.0 g, 4.70 mmol), 3-pyridinylboronic acid (0.75 g, 6.11 mmol) and tetrakis(triphenylphosphine)palladium(0) (5 mol %) in 120 mL of toluene and 30 mL of ethanol was dissolved. To the reaction mixture was added a solution of potassium carbonate (2 M, 40 mL) dropwise slowly and refluxed under argon for 24 h. After being cooled to ambient temperature, the reaction mixture was extracted with toluene and water. The organic layer was evaporated with a rotary evaporator. The product was purified by column chromatography using ethyl acetate and chloroform mixture (1:9) and 3PySDP as a white solid product was obtained (0.1 g, 5.2%). Yield 5.2%. ¹H NMR (300 MHz, CDCl₃, ppm): δ 8.83 (s, 2H), 8.58-8.57 (m, 2H), 7.86-7.83 (d, 2H), 7.48-6.94 (m, 11H), 6.68-6.62 (m, 4H), 5.40-5.34 (m, 2H), 4.65-4.61 (m, 2H). ¹³C NMR (500 MHz, CDCl₃, ppm): δ 148.3, 145.5, 142.2, 141.1, 138.6, 137.4, 134.1, 132.7, 132.2, 131.7, 130.8, 128.7, 128.0, 127.7, 127.4, 127.0, 126.8, 126.4, 126.2, 125.9, 123.5, 58.1, 47.2, 38.9; MALDI/TOF-MS 423 ([M+H]⁺).

### Example 9

### Preparation of 5,5-Phenyl(4-(pyridine-4-yl)phenyl)-9H-dibenzosuberane (4PySDP)

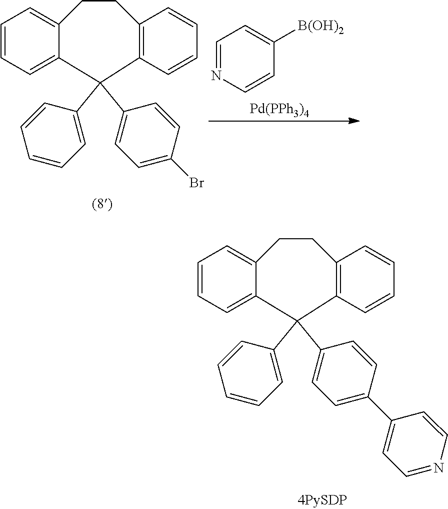

A mixture of 5,5-(4-bromophenyl)(phenyl)-9H-dibenzosuberane (8′) (2.0 g, 4.70 mmol), 4-pyridinylboronic acid (0.75 g, 6.11 mmol) and tetrakis(triphenylphosphine)palladium(0) (5 mol %) in 30 mL of THF was dissolved. To the reaction mixture was added a solution of potassium carbonate (2 M, 30 mL) dropwise and refluxed under argon for 24 h. After being cooled to ambient temperature, the reaction mixture was extracted with toluene and water. The organic layer was evaporated with a rotary evaporator. The product was purified by column chromatography using a mixture solvent (methylene chloride/n-hexane=1:1 and then ethyl acetate and chloroform=1:9) and 4PySDP as white powder was obtained (0.8 g, 42%). Yield 42%. ¹H NMR (300 MHz, CDCl₃, ppm): δ 8.70-8.64 (m, 4H), 7.74-6.80 (m, 13H) 6.59-6.57 (d, 2H), 6.07-6.04 (d, 2H) 5.43˜5.41 (m, 2H), 5.12-5.09 (m, 2H). ¹³C NMR (500 MHz, CDCl₃, ppm): δ 150.3, 147.9, 146.5, 144.3, 141.6, 141.2, 138.4, 137.3, 135.7, 132.2, 131.5, 130.5, 128.3, 128.0, 127.6, 127.5, 127.2, 126.7, 121.4, 58.2, 46.6, 36.2; MALDI/TOF-MS 423 ([M+H]⁺).

### Example 10

### Preparation of 5,5-(4-Biphenyl)(phenyl)-9H-dibenzosuberane (PSDP)

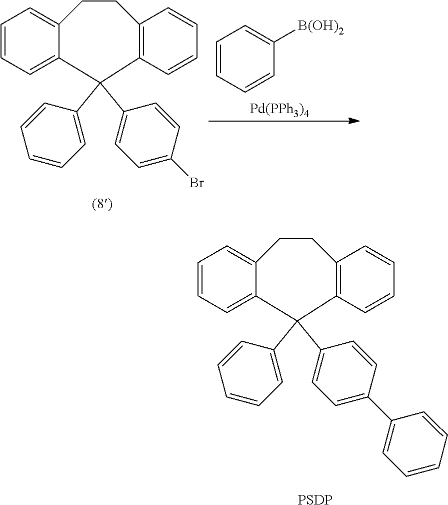

A mixture of 5,5-(4-bromophenyl)(phenyl)-9H-dibenzosuberane (8′) (2.0 g, 4.70 mmol), phenylboronic acid (0.74 g, 6.11 mmol) and tetrakis(triphenylphosphine)palladium(0) (5 mol %) in 30 mL of THF was dissolved. To the reaction mixture was added a solution of potassium carbonate (2 M, 30 mL) dropwise and refluxed under argon for 24 h. After being cooled to ambient temperature, the reaction mixture was extracted with ethyl acetate and water. The organic layer was evaporated with a rotary evaporator. The product was purified by column chromatography using n-hexane and PSDP as a white solid product was obtained (1.1 g, 55%). Yield 55%. ¹H NMR (300 MHz, CDCl₃, ppm): δ 7.60-6.80 (m, 18H), 6.58-6.55 (d, 2H), 6.05-6.03 (d, 2H), 5.43˜5.37 (m, 2H), 5.13˜5.10 (m, 2H). ¹³C NMR (500 MHz, CDCl₃, ppm): δ 144. 5, 144.3, 141.7, 141.5, 140.9, 138.8, 138.7, 137.4, 132.9, 132.6, 132.2, 131.4, 130.5, 128.7, 127.9, 127.4, 127.0, 126.8, 126.5, 126.4, 123.8, 58.1, 46.6, 36.2; MALDI/TOF-MS 423 ([M+H]⁺).

### Example 11

### Preparation of 3DPySDP

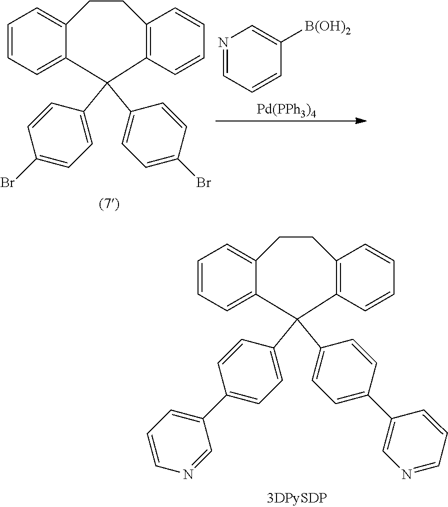

A mixture of 5,5-Bis(4-bromophenyl)-9H-dibenzosuberane (7′) (3.50 g, 7.01 mmol), 3-pyridinylboronic acid (2.58 g, 21.04 mmol) and tetrakis(triphenylphosphine)palladium(0) (5 mol %) in 120 mL of toluene and 30 mL of ethanol was dissolved under argon. To the reaction mixture was added a solution of potassium carbonate (2 M, 40 mL) dropwise slowly and was refluxed under argon for 24 h at 120° C. After being cooled to ambient temperature, the reaction mixture was extracted with dichloromethane and water. The organic layer was evaporated with a rotary evaporator. The product was purified by column chromatography using ethyl acetate and chloroform mixture (10:90) and 3DPySDP as a white solid product was obtained (71% yield). Yield 71%. ¹H NMR (300 MHz, CDCl₃, ppm): δ 8.88 (s, 2H), 8.62 (s, 2H), 7.91-7.89 (m, 2H), 7.64-7.22 (m, 16H), 6.96-6.81 (m, 2H), 5.97 (s, 4H). ¹³C NMR (500 MHz, CDCl₃, ppm): δ 150.3, 148.3, 148.2, 143.2, 140.8, 137.2, 136.0, 134.3, 130.3, 129.5, 128.4, 127.2, 126.7, 126.4, 123.6, 123.0, 56.1, 49.7; MALDI/TOF-MS 501 ([M+H]⁺).

### Example 12

### Preparation of 4DPySDP

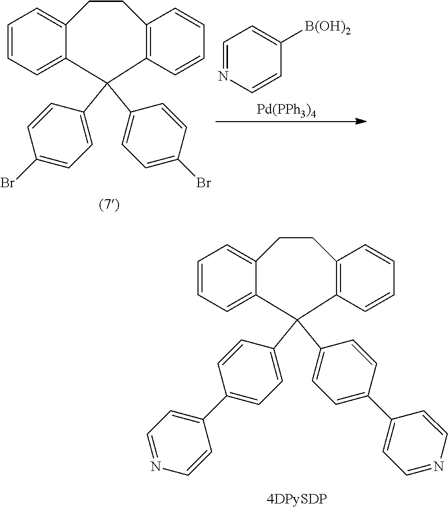

A mixture of 5,5-bis(4-bromophenyl)-9H-dibenzosuberane (7′) (2.0 g, 3.96 mmol), 4-pyridinylboronic acid (1.21 g, 9.92 mmol) and tetrakis(triphenylphosphine)palladium(0) (5 mol %) in 30 mL of tetrahydrofuran was dissolved under argon. To the reaction mixture was added a solution of potassium carbonate (2 M, 30 mL) dropwise slowly and was refluxed under argon for 24 h at 120° C. After being cooled to ambient temperature, the reaction mixture was extracted with dichloromethane and water. The organic layer was evaporated with a rotary evaporator. The product was purified by column chromatography using ethyl acetate and chloroform mixture (10:90) and 4DPySDP as a white solid product was obtained. Yield 71%. ¹H NMR (300 MHz, CDCl₃, ppm): δ 8.68-8.62 (m, 4H), 7.73-7.63 (m, 4H), 7.53-6.78 (m, 16H), 5.99 (s, 4H). ¹³C NMR (500 MHz, CDCl₃, ppm): δ 150.3, 149.6, 148.7, 147.8, 144.2, 140.6, 136.4, 131.7, 130.4, 129.8, 128.5, 127.9, 127.2, 126.8, 126.5, 124.7, 121.4, 53.0, 43.9; MALDUTOF-MS 501 ([M+H]⁺).

### Example 13

### Preparation of DPSDP

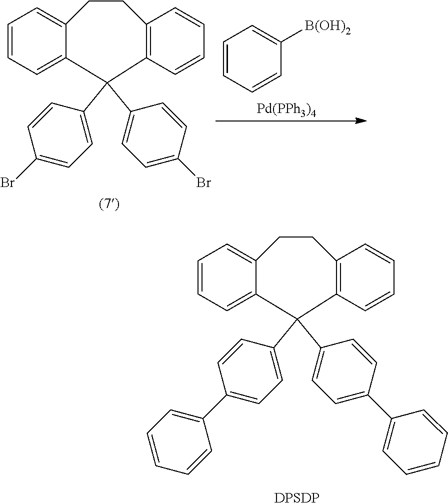

A mixture of 5,5-bis(4-bromophenyl)-9H-dibenzosuberane (7′) (2.0 g, 3.96 mmol), phenylboronic acid (1.2 g, 9.92 mmol) and tetrakis(triphenylphosphine)palladium(0) (5 mol %) in 30 mL of THF was dissolved. To the reaction mixture was added a solution of potassium carbonate (2 M, 30 mL) dropwise and refluxed under argon for 24 h. After being cooled to ambient temperature, the reaction mixture was extracted with ethyl acetate and water. The organic layer was evaporated with a rotary evaporator. The product was purified by column chromatography using n-hexane and DPSDP as a white solid product was obtained (1.3 g, 65%). Yield 65%. ¹H NMR (300 MHz, CDCl₃, ppm): δ 7.68-6.98 (m, 26H), 6.03 (s, 4H). ¹³C NMR (500 MHz, CDCl₃, ppm): δ 142.6, 140.9, 139.3, 137.1, 135.8, 130.4, 130.2, 130.1, 128.8, 128.1, 127.7, 127.6, 127.4, 127.2, 127.1, 126.6, 126.3, 52.9, 49.7; MALDI/TOF-MS 499 ([M+H]⁺).

### Example 14

### Preparation of 2,7-DPySDF

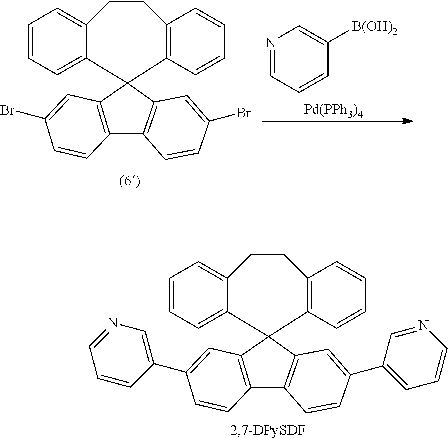

A mixture of 2,7-dibromo-spiro[fluorene-9,5′-dibenzosuberane] (6′) (2.5 g, 6.97 mmol), 3-pyridinylboronic acid (1.83 g, 14.9 mmol) and tetrakis(triphenylphosphine)palladium(0) (5 mol %) in 120 mL of toluene and 30 mL of ethanol was dissolved. To the reaction mixture was added a solution of potassium carbonate (2 M, 40 mL) dropwise slowly and then was refluxed under argon for 24 h. After cooling to ambient temperature, the reaction mixture was extracted with toluene and water. The organic layer was evaporated with a rotary evaporator. The product was purified by column chromatography using ethyl acetate and chloroform mixture (10:90) and 2,7-DPySDF as a white solid product was obtained (1.1 g, 31% yield). Yield 31%. ¹H NMR (300 MHz, CDCl₃, ppm): δ 8.85-8.82 (m, 2H), 8.57-8.43 (m, 2H), 8.08-7.02 (m, 16H), 6.59-6.36 (m, 2H), 5.77 (s, 4H). ¹³C NMR (500 MHz, CDCl₃, ppm): δ 150.3, 149.3, 148.4, 140.5, 139.7, 137.4, 137.1, 134.3, 130.2, 128.6, 128.2, 127.8, 126.7, 126.4, 123.9, 123.5, 120.88, 56.1, 49.8; MALDI/TOF-MS 500 ([M+H]⁺).

### Example 15

### Preparation of 3,6-dibromo-spiro[fluorene-9,5′-dibenzosuberane]

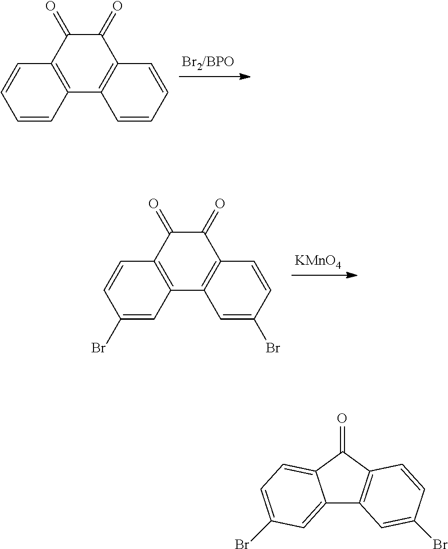

3,6-Dibromophenantrenequinone To a mixture of phenanthrenequinone (7.0 g, 33.6 mmol) and benzoyl peroxide (0.8 g, 3.36 mmol) in 100 mL nitrobenzene was added dropwise bromine (4.19 mL, 84.0 mmol). After complete addition the reaction mixture was heated at 110° C. during 12 hours. The 3,6-dDibromophenantrenequinone product was washed extensively with hexane and used without further purification. Yield: 8.0 g (65%). ¹H NMR (300 MHz, CDCl₃): δ 8.17 (d, 2H), 8.13 (d, 2H), 7.72 (dd, 2H). ¹³C NMR (500 MHz, CDCl₃): δ 178.3, 136.2, 134.4, 133.2, 132.3, 130.7, 127.5; GC-MS(EI) 366 ([M+H⁺]).

3,6-Dibromofluorenone A mixture of KMnO₄ (12.45 g, 222.9 mmol) and KOH (117 g, 741 mmol) in 400 mL water was heated to reflux. Then 3,6-dibromophenanthrenequinone (8.0 g, 21.85 mmol) was added at once. Heating was continued for 4 hours. The mixture was allowed to cool to room temperature and dichloromethane was added. The organic layer was separated, dried with MgSO₄, filtered and concentrated. The solid material was transferred to Soxhlet extractor and extracted with toluene for 24 hours. Yellow crystals of pure 3,6-dibromofluorenone product were isolated. Yield 67° A). ¹H NMR (300 MHz, CDCl₃): δ 7.71 (d, 2H), 7.58 (d, 2H), 7.52 (dd, 2H). ¹³C NMR (500 MHz, CDCl₃): δ 145.3, 133.7, 133.2, 130.3, 126.1, 124.8; GC-MS(EI) 338 ([M+H⁺]).

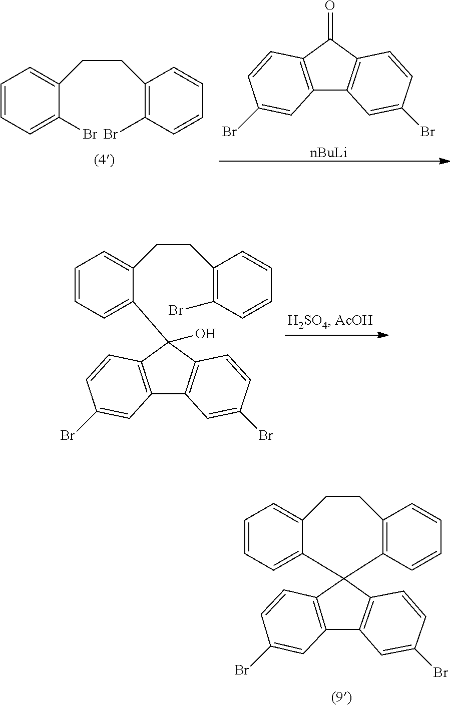

To a 250 mL two-necked flask was placed a solution of 1,2-bis(2-bromophenyl)ethane (4′) (4.19 g, 12.32 mmol) in THF (50 mL). The reaction flask was cooled to −78° C. and n-butyllithium (2.5 M in n-hexane, 5.91 mL) was added dropwise slowly. The whole solution was stirred at this low temperature for 2 h, followed by the addition of a solution of 3,6-dibromo-9-fluorenone (5.0 g, 14.78 mmol) in THF (400 mL) under an argon atmosphere. The resulting mixture was gradually warmed to ambient temperature and quenched by adding saturated, aqueous NaHCO₃ (300 mL). The mixture was extracted with dichloromethane. The combined organic layers were dried over MgSO₄, filtered and evaporated under reduced pressure yielding a yellow powdery product. The crude residue was placed in another two-necked flask and dissolved in acetic acid (100 mL). A catalytic amount of aqueous H₂SO₄ (10 mol %) was then added and the whole solution was refluxed for 12 h. After cooling to ambient temperature, purification by silica gel chromatography using n-hexane as an eluent gave 3,6-dibromo-spiro[fluorene-9,5′-dibenzosuberane] (9′) as a white powder. Yield 1.1 g, 16%. ¹H NMR (300 MHz, CDCl₃, ppm): δ 7.96-7.00 (m, 10H), 6.48-6.26 (m, 4H), 5.53 (s, 4H). ¹³C NMR (500 MHz, CDCl₃, ppm): δ 142.0, 141.1, 135.8, 130.8, 126.8, 123.4, 121.4, 120.3, 58.9, 42.2; MALDI/TOF-MS 503 ([M+H]⁺).

### Example 16

### Preparation of 3,6-DPySDF

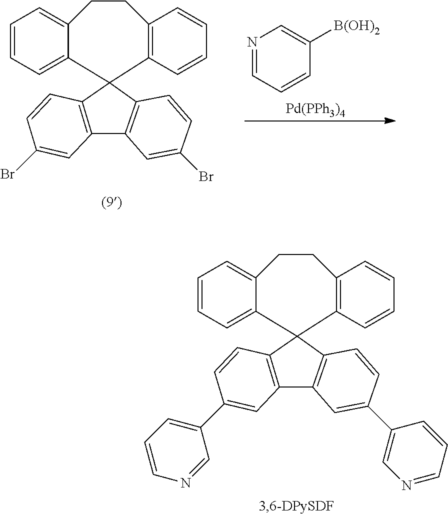 [SMILES: Brc1ccc2c(c1)-c1cc(Br)ccc1C21c2ccccc2CCc2ccccc21 ; OB(O)c1cccnc1 ; c1cncc(-c2ccc3c(c2)-c2cc(-c4cccnc4)ccc2C32c3ccccc3CCc3ccccc32)c1 ; "3,6-DPySDF" ; "Pd(PPh3)4"]

A mixture of 3,6-dibromo-spiro[fluorene-9,5′-dibenzosuberane] (9′) (1.0 g, 1.90 mmol), 3-pyridinylboronic acid (0.61 g, 4.97 mmol) and tetrakis(triphenylphosphine)palladium(0) (5 mol %) in 30 mL of tetrahydrofuran was dissolved. To the reaction mixture was added a solution of potassium carbonate (2 M, 30 mL) dropwise slowly and then was refluxed under argon for 24 h. After cooling to ambient temperature, the reaction mixture was extracted with toluene and water. The organic layer was evaporated with a rotary evaporator. The product was purified by column chromatography using ethyl acetate and chloroform mixture (40:10) and a white solid product was obtained (0.61 g, 64% yield). Yield 64%. ¹H NMR (300 MHz, CDCl₃, ppm): δ 8.97-8.92 (d, 2H), 8.79 (s, 1H), 8.65 (s, 2H), 8.38 (s, 1H), 8.16-7.83 (m, 6H), 7.55-7.05 (m, 8H), 6.58-6.35 (m, 2H), 5.73 (s, 4H). ¹³C NMR (500 MHz, CDCl₃, ppm): δ 150.3, 150.0, 148.1, 141.6, 137.4, 136.8, 134.7, 130.5, 130.1, 128.5, 127.9, 127.4, 127.0, 125.9, 123.7, 123.2, 118.8, 55.6, 49.3; MALDI/TOF-MS 500 ([M+H]⁺).

### Example 17

### Molecular Simulation of 10,11-DPSDF and 10,11-DQSDF

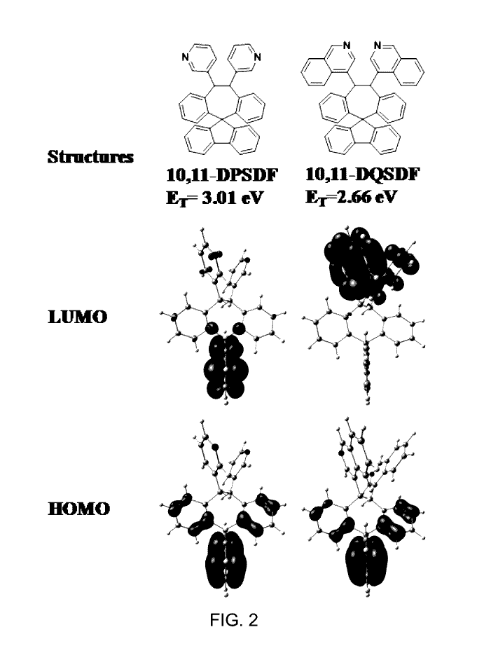

Molecular simulation results of 10,11-DPSDF and 10,11-DQSDF are shown in FIG. 2. The ab initio calculations were performed using a suite of Gaussian 03 programs and the molecular structures of 10,11-DPSDF and 10,11-DQSDF were fully optimized by density functional theory (DFT) using Beck's three parameterized Lee-Yang-Parr exchange functional (B3LYP) with 6-31 G* basis sets. The HOMO orbitals are distributed over the whole structure of 10,11-DPSDF and 10,11-DQSDF. This indicates that HOMO levels of 10,11-DPSDF and 10,11-DQSDF are determined largely by the fluorene structure. The LUMO orbitals of the pyridine substituents are dispersed in the fluorene and suberane moieties. However, the molecular structure of quinoline substituted compounds, and the LUMO orbital was distributed into the quinoline groups. By this mean, the LUMO mostly depends on the substituents with strong electron transport properties, leading to the LUMO level for electron injection.

The calculated data of triplet energy and HOMO/LUMO energy levels are shown in Table 1. The calculated results indicate that the triplet energy of 10,11-DPSDF and 10,11-DQSDF are 3.01 eV and 2.66 eV, respectively.

TABLE 1
Calculated energy levels and
E_{T} of 10,11-DPSDF and 10,11-DQSDF.

| | HOMO (eV) | LUMO (eV) | E_{g} (eV) | E_{T} (eV) |
|---|---|---|---|---|
| 10,11-DPSDF | −5.79 | −0.97 | 4.81 | 3.01 |
| 10,11-DQSDF | −5.76 | −1.48 | 4.27 | 2.66 |

### Example 18

### Photophysical Properties of 2PySDP, 3PySDP, 4PySDP and PSDP

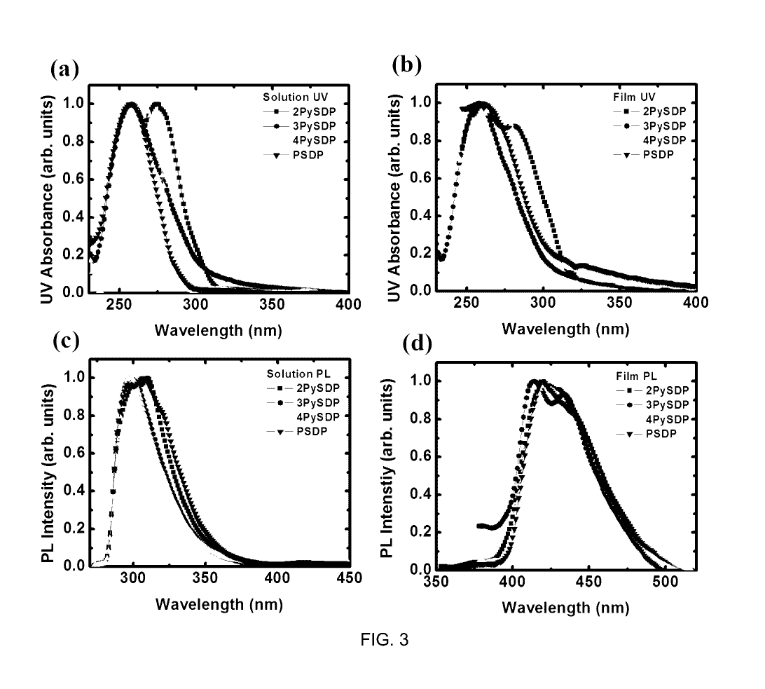

UV-vis optical absorption and photoluminescence (PL) spectra of 2PySDP, 3PySDP, 4PySDP and PSDP in dilute THF solution (10-5 M) and thin films are shown in FIG. 3. Photophysical properties of the 2PySDP, 3PySDP, 4PySDP and PSDP are summarized in Table 2. The absorption peak of 2PySDP was 275 nm in dilute THF solution and 259 nm with a shoulder peak at 280 nm in thermally evaporated thin films. In the case of 3PySDP and PSDP, the absorption maximum (λₘₐₓ^{abs}) values were 258 nm and 257 nm, respectively, in solution as well as thin films. The absorption peak of 4PySDP was found at 264 nm in solution and at 268 nm in thin film. The similarity of the λₘₐₓ^{abs} values of these compounds originated from the same core molecular structure: 5,5′-bis(phenyl)-9H-dibenzosuberane. The optical band gaps (E_{g}^{opt}) of the four compounds determined from the absorption edge of the thin film spectra was found to be 3.88-4.00 eV (Table 2). The PL emission peak of 3PySDP and 4PySDP in solution were observed at 298 nm whereas 2PySDP and PSDP showed at 310 nm and 307 nm, respectively. Thin film PL emission peaks were found in the 414 to 425 nm range. The solid-state emission spectra were dramatically red shifted from the solution spectra, which implied high intermolecular interactions.

TABLE 2
Photophysical, thermal, and electrochemical properties
of 2PySDP, 3PySDP, 4PySDP and PSDP.

| | | 2PySDP | 3PySDP | 4PySDP | PSDP |
|---|---|---|---|---|---|
| λₘₐₓ^{abs} | Solution^{a} | 275 (4.64) | 258 (4.97) | 264 (4.87) | 257 (5.04) |
| (nm) | (log ∈) | | | | |
| | Thin film^{c} | 259, 280 | 258 | 268 | 258 |
| λₘₐₓ^{em} | Solution^{a} | 310 | 298 | 298 | 307 |
| (nm) | Thin film^{c} | 421 | 414 | 425 | 419 |

| | | | | |
|---|---|---|---|---|
| E_{g}^{opt}(eV)^{d} | 3.88 | 4.00 | 3.93 | 3.97 |
| E_{T} (eV) | 2.87 | 2.85 | 2.84 | 2.80 |
| LUMO (eV) | −2.43 | −2.33 | −2.4 | −2.32 |
| HOMO (eV) | −6.3 | −6.33 | −6.33 | −6.29 |
| Tₘ (° C.) | 170 | 154 | 155 | 200 |
| T_{d} (° C.) | 347 | 349 | 329 | 342 |

^{a}The absorption and emission spectra in dilute THF solution (10⁻⁵ M).
^{b}log ∈ calculated at λₘₐₓ^{abs}.
^{c}The thin films were thermally evaporated.
^{d}Calculated from the absorption band edge of the thin film.

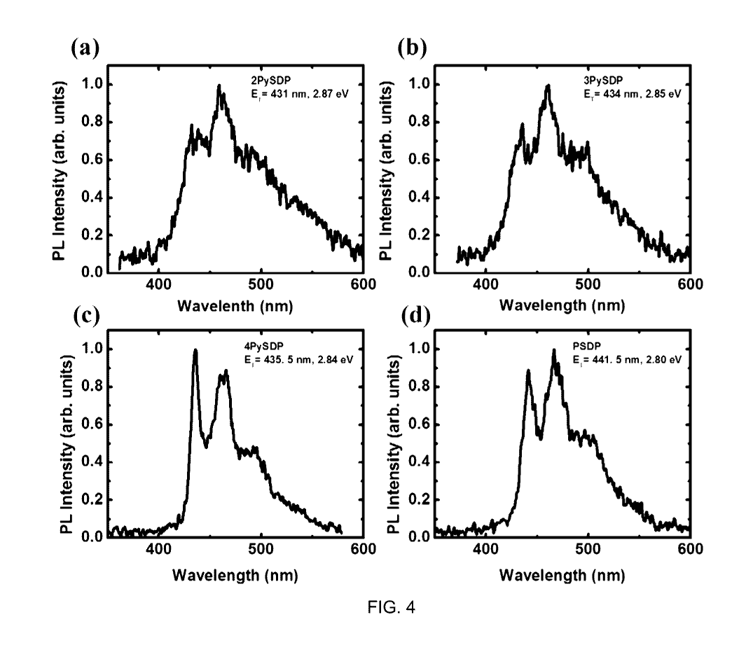

Phosphorescence spectra were obtained at 77 K to measure the triplet energies of the compounds as shown in FIG. 4. Phosphorescent PL spectra were recorded on a Photon Technology International (PTI) Inc. Model QM 2001-4 spectrofluorimeter. Triplet energy values of the dibenzosuberane-based materials were estimated from the highest energy peaks in phosphorescent spectra. Each sample was prepared in dilute 2-methyltetrahydrofuran solution with concentrations of 3˜5 mg/mL. The excitation wavelength was fixed at the wavelength which showed the maximum absorbance. A delayed detection time of 500 μs and 100˜150 Hz of chopper frequency was set in order to measure phosphorescence exclusively. The actual PL intensity value of the dibenzosuberane-based materials were in the range of 1000 to 2000 photon counts (maximum limit of detector=2500 counts). The triplet energies of 2PySDP, 3PySDP, 4PySDP, PDSP were determined from the highest energy peak of the low temperature phosphorescent PL spectra and found to be 2.80-2.87 eV (see Table 2). The triplet energies of the four compounds are high enough to confine the triplet excitons of blue Flrpic (E_{T}=2.7 eV). The results demonstrate that these four materials with high triplet energy are very promising for blue phosphorescent OLEDs (PhOLEDs).

### Example 19

### Thermal Properties of 2PySDP, 3PySDP, 4PySDP and PSDP

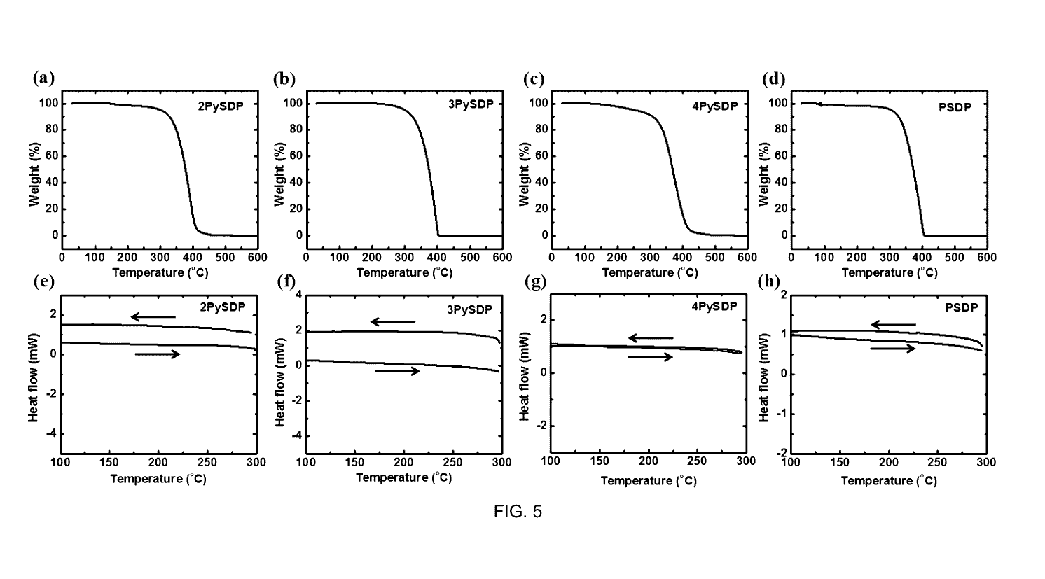

Thermal properties of 2PySDP, 3PySDP, 4PySDP and PSDP were characterized by differential scanning calorimetry (DSC) and thermogravimetric analysis (TGA). The TGA and DSC thermograms are shown in FIG. 5. Glass transition temperatures (T_{g}) or melting temperatures (Tₘ) from DSC scans in the 30-300° C. range could not be observed so a melting point measuring machine was used to observe the melting point (Tₘ). The onset decomposition temperatures (T_{d}) of the compounds were high (>329° C.), which demonstrates their thermal robustness. This means that these compounds have amorphous structure and are indeed thermally stable.

### Example 20

### Electrochemical Properties of 2PySDP, 3PySDP, 4PySDP and PSDP

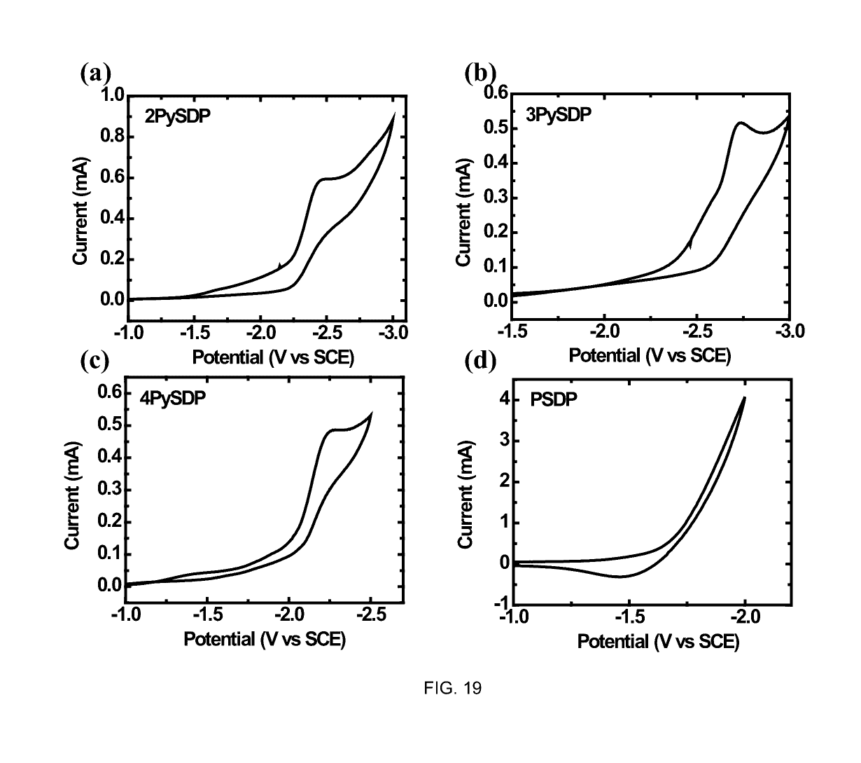

The HOMO/LUMO energy levels of 2PySDP, 3PySDP, 4PySDP and PSDP were estimated from cyclic voltammetry (CV) and in some cases in combination with the absorption edge optical band gap. The cyclic voltammograms are shown in FIG. 19. The HOMO/LUMO energy levels of 2PySDP, 3PySDP, 4PySDP and PSDP are summarized in Table 2 hereinabove. The LUMO levels of 2PySDP, 3PySDP, 4PySDP and PSDP were obtained from the onset reduction potential of the CV, giving LUMO levels of −2.43, −2.33, −2.4 and −2.32 eV, respectively, which are much higher than that of well-known electron transport material tris(8-hydroxyquinoline)aluminum (Alq₃) (−3.0 eV) and similar to well-known hole-blocking material 2,9-dimethyl-4,7-diphenyl-phenathroline (BCP) (−2.4 eV). The HOMO levels of the four compounds were found to be −6.3, −6.33, −6.33 and −6.29 eV, respectively, which were estimated from the difference between LUMO level and the optical band gap. It may also be possible to use these compounds as host materials because the HOMO/LUMO energy levels of the four molecules are very similar with those of N,N-dicarbazolyl-3,5-benzene (mCP) (−6.1 eV/−2.4 eV), which is a very well-known host material in highly efficient PhOLEDs.

### Example 21

### Photophysical Properties of 3DPySDP, 4DPySDP and DPSDP

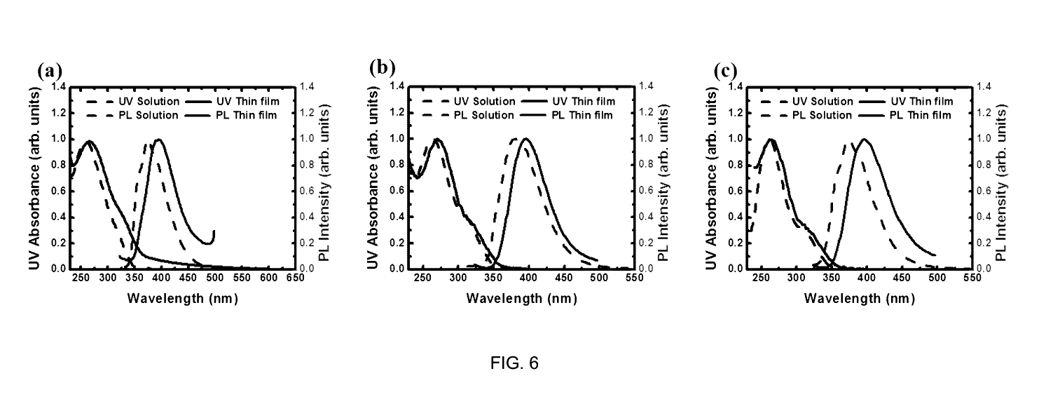

Optical absorption and photoluminescence (PL) spectra of the 3DPySDP, 4DPySDP and DPSDP in dilute THF solution (10⁻⁵ M) and in thin films are shown in FIG. 6. The solid state absorption and PL emission spectra of 3DPySDP, 4DPySDP and DPSDP were obtained from thermally evaporated thin films. The key numerical values of the photophysical properties of these compounds, including absorption maximum (λₘₐₓ^{abs}), molar absorption coefficient (log ε), PL emission maximum (λₘₐₓ^{em}) and optical band gap (E_{g}^{opt}) are summarized in Table 3. A strong solution absorption peak was observed between 254 nm and 263 nm which is assigned to the absorption of the spirodibenzosuberane unit in the molecules. Similar absorption spectra were observed in the three compounds due to the common spirodibenzosuberane core in the molecules. The absorption peak of 3DPySDP, 4DPySDP and DPSDP were observed 266, 271 and 262 nm, respectively, as thin films. The PL emission maximum (λₘₐₓ^{em}) of 3DPySDP, 4DPySDP and DPSDP was observed at 375, 381 and 374 nm, respectively in THF solution. The emission maxima in the films are red shifted around 20 nm from the solution spectra. Optical band gaps of 3DPySDP, 4DPySDP and DPSDP were estimated from the absorption edge of the UV-Vis spectra, revealing E_{g}^{opt} of 3.4, 3.44 and 3.46 eV, respectively.

TABLE 3
Photophysical, electrochemical, and thermal
properties of 3DPySDP, 4DPySDP and DPSDP.

| | | 3DPySDP | 4DPySDP | DPSDP |
|---|---|---|---|---|
| λₘₐₓ^{abs} | Solution^{a} | 258 (4.45) | 263 (4.80) | 260 (4.56) |
| (nm) | (log ∈)^{b} | | | |
| | Thin film^{c} | 266 | 271 | 262 |
| λₘₐₓ^{em} | Solution^{a} | 375 | 381 | 374 |
| (nm) | Thin film^{c} | 395 | 396 | 397 |

| | | | |
|---|---|---|---|
| E_{g}^{opt}(eV)^{d} | 3.4 | 3.44 | 3.46 |
| E_{T} (eV) | 3.0 | 3.26 | 3.29 |
| LUMO (eV) | −2.7 | −2.51 | −2.48 |
| HOMO (eV) | −6.1 | −5.95 | −5.94 |
| T_{g} (° C.) | None | 112 | None |
| Tₘ (° C.) | 157 | 177 | 152 |
| T_{d} (° C.) | 418 | 382 | 404 |

^{a}The absorption and emission spectra in dilute THF solution (10⁻⁵ M).
^{b}log ∈ calculated at λₘₐₓ^{abs}.
^{c}Thin films were thermally evaporated.
^{d}Calculated from the thin film absorption band edge.

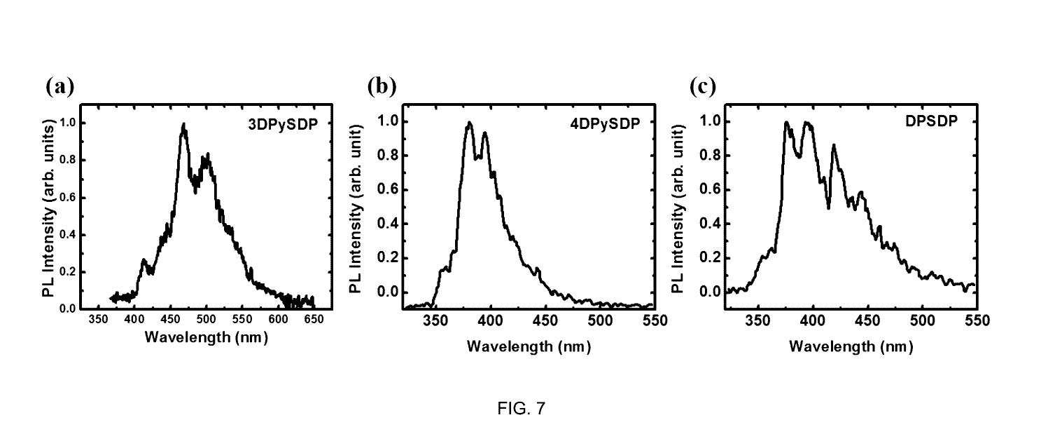

The triplet energy (E_{T}) of 3DPySDP, 4DPySDP and DPSDP was estimated from the shortest wavelength emission peak of the phosphorescence spectrum obtained at low temperature (77K) in dilute 2-methyl tetrahydrofuran solution. The excitation wavelength was fixed at the wavelength which showed the maximum absorbance. A delayed detection time of 500 μs and 100˜150 Hz of chopper frequency was set in order to measure phosphorescence exclusively. The actual PL intensity value of the dibenzosuberane-based materials were in the range of 1000 to 2000 photon counts (maximum limit of detector=2500 counts). The phosphorescent spectra of 3DPySDP, 4DPySDP and DPSDP are shown in FIG. 7. The measured triplet energies of the three compounds are given in Table 3 above. 3DPySDP, 4DPySDP and DPSDP with E_{T} values over 3.0 eV are high enough to confine the triplet excitons of Flrpic triplet emitter with E_{T} of 2.7 eV. The measured triplet energy values of these compounds are much higher than those of commercial electron transport materials, such as 2,9-dimethyl-4,7-diphenyl-1,10-phenanthroline (BCP) (E_{T}=2.5 eV) and 1,3,5-tri(m-pyrid-3-yl-phenyl) (TmPyPB) (E_{T}=2.78 eV).

### Example 22

### Thermal Properties of 3DPySDP, 4DPySDP and DPSDP

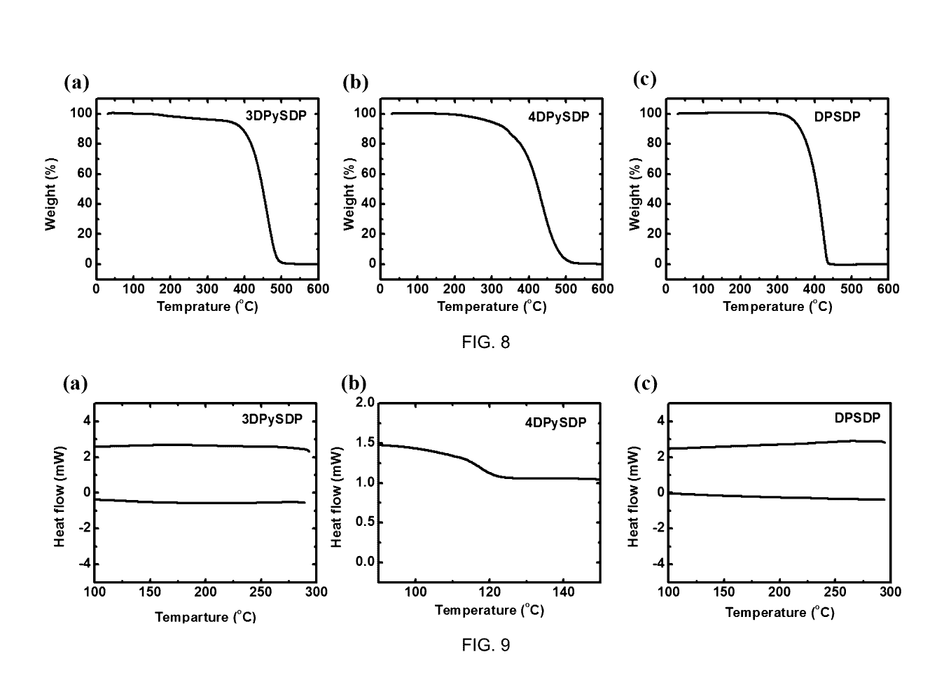

Thermal properties of 3DPySDP, 4DPySDP and DPSDP were characterized by thermogravimetric analysis (TGA) and differential scanning calorimetry (DSC). TGA and DSC curves of these compounds are shown in FIG. 8 and FIG. 9, respectively. Numerical values extracted from the TGA and DSC scans are given in Table 3 above. Three distinct transitions were observed in the second-heating/cooling DSC scans of 3DPySDP, 4DPySDP and DPSDP. Both 3DPySDP and DPSDP did not show glass transition temperature (T_{g}) whereas 4DPySDP showed a T_{g} at 112° C. A melting point measuring machine was used to observe the melting point (Tₘ). The melting points (Tₘ) of 3DPySDP, 4DPySDP and DPSDP were found to be 157, 177 and 152° C., respectively. These compounds showed onset decomposition temperature (T_{d}) in the range of 382 to 418° C. demonstrating their thermal robustness. A complete thermal decomposition with remained weight ratio of zero % suggests that the materials can be readily evaporated to form thin films.

### Example 23

### Electrochemical Properties of 3DPySDP, 4DPySDP and DPSDP

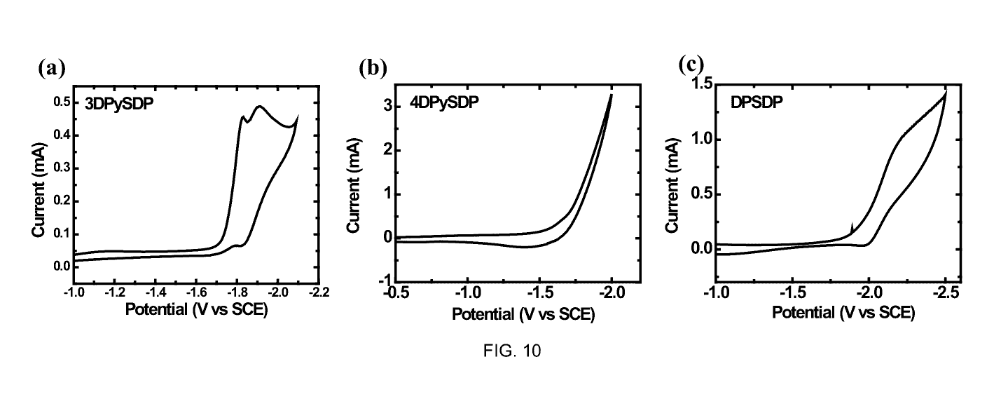

Electronic structure (LUMO/HOMO energy levels) of 3DPySDP, 4DPySDP and DPSDP was studied by cyclicvoltammetry (CV). The cyclic voltammograms of the ETMs in solution are shown in FIG. 10. The reduction CVs of the three materials were not reversible. The LUMO levels of 3DPySDP, 4DPySDP and DPSDP were found to be −2.7, −2.51 and −2.48 eV, respectively. Oxidation was not observed for any of the compounds. The HOMO levels of 3DPySDP, 4DPySDP and DPSDP were found to be −6.1, −5.95 and −5.94 eV, respectively, estimated from the optical band gap (E_{g}^{opt}). The results suggest that these compounds have good exciton as well as hole blocking properties for blue PhOLEDs.

### Example 24

### Photophysical Properties of 2,7-DPySDF and 3,6-DPySDF

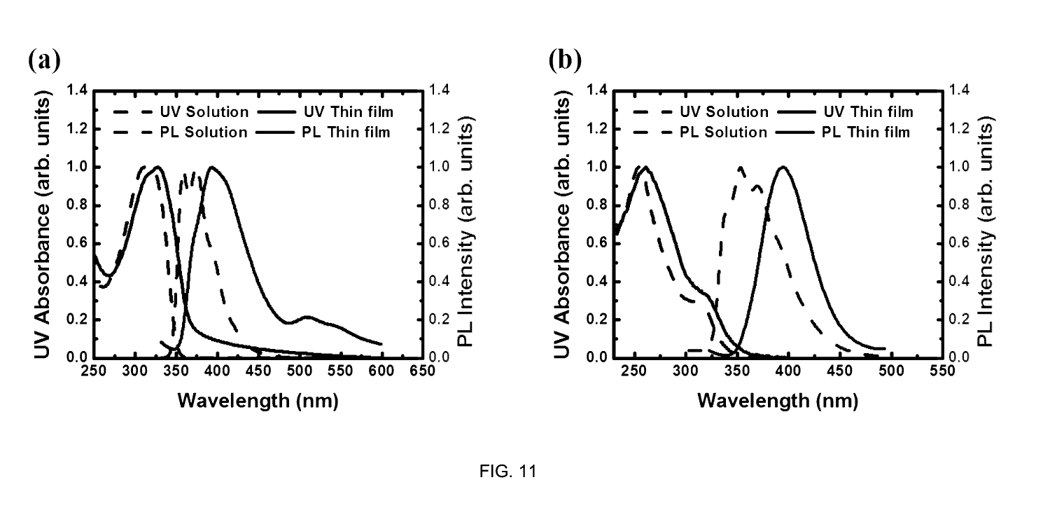

Optical absorption and photoluminescence (PL) spectra of 2,7-DPySDF and 3,6-DPySDF in dilute toluene solution (10⁻⁶ M) and thin films are shown in FIG. 11. Photophysical properties of 2,7-DPySDF and 3,6-DPySDF are summarized in Table 4. The absorption peaks of 2,7-DPySDF and 3,6-DPySDF are observed at 311 nm and 254 nm in THF solution. The PL emission spectra of 2,7-DPySDF and 3,6-DPySDF showed maximum peak around 355 nm with a shoulder peak around 370 nm in solution and the PL emission maximum peak at 395 nm in thin films. The optical band gaps of the two compounds were 3.4 and 3.53 eV, respectively, determined from the absorption edges of the thin films.

TABLE 4
Photophysical, electrochemical, and thermal
properties of 2,7-DPySDF and 3,6-DPySDF.

| | | 2,7-DPySDF | 3,6-DPySDF |
|---|---|---|---|
| λₘₐₓ^{abs} | Solution ^{a} (log ∈)^{b} | 311 (4.55) | 254 (4.89) |
| (nm) | Thin film^{c} | 327 | 261 |
| λₘₐₓ^{em} | Solution ^{a} | 358, 375 | 353, 370 |
| (nm) | Thin film ^{c} | 393.5 | 395 |

| | | |
|---|---|---|
| E_{g}^{opt}(eV)^{d} | 3.4 | 3.53 |
| E_{T} (eV) | 2.45 | 3.17 |
| LUMO (eV) | −2.61 | −2.71 |
| HOMO (eV) | −6.01 | −6.24 |
| T_{g} (° C.) | 100 | 130 |
| Tₘ (° C.) | 163 | 191 |
| T_{d} (° C.) | 415 | 439 |

^{a}The solution absorption and emission spectra in dilute THF solution (5 × 10⁻⁵ M).
^{b}log ∈ calculated at λₘₐₓ^{abs}.
^{c}The thin films were thermally evaporated.
^{d}Calculated from the thin film absorption band edge.

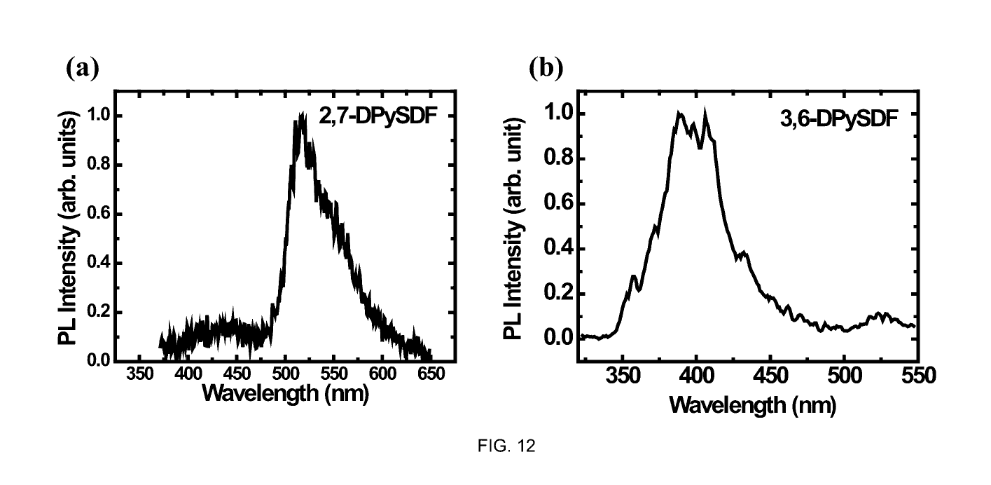

The phosphorescence spectra were also obtained at 77 K to measure the triplet energy of the compounds as shown in FIG. 12. Each sample was prepared in dilute 2-methyltetrahydrofuran solution with concentrations of 3˜5 mg/mL. The excitation wavelength was fixed at the wavelength which showed the maximum absorbance. A delayed detection time of 500 μs and 100˜150 Hz of chopper frequency was set in order to measure phosphorescence exclusively. The actual PL intensity value of the dibenzosuberane-based materials were in the range of 1000 to 2000 photon counts (maximum limit of detector=2500 counts). The triplet energy of 2,7-DPySDF and 3,6-DPySDF was also determined from the highest energy peak of the low temperature PL spectrum and found to be 2.45 eV and 3.17 eV, respectively. In the case of 3,6-DPySDF, the triplet energy is high enough to confine the triplet excitons of Flrpic (ET=2.7 eV). The HOMO/LUMO and triplet energy levels of are summarized in Table 4.

### Example 25

### Thermal Properties of 2,7-DPySDF and 3,6-DPySDF

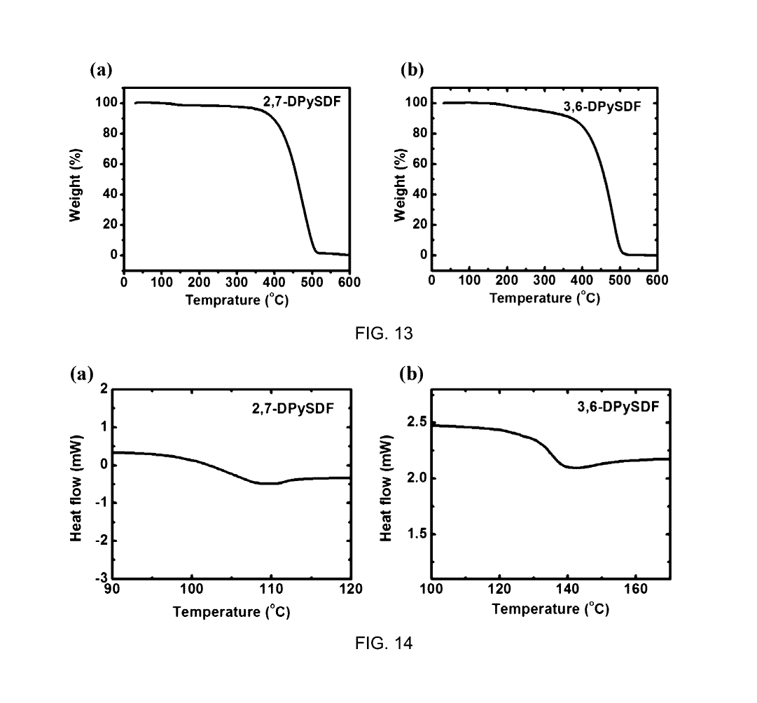

TGA and DSC curves of the 2,7-DPySDF and 3,6-DPySDF are shown in FIG. 13 and FIG. 14, respectively. Thermal properties of the 2,7-DPySDF and 3,6-DpySDF were characterized by differential scanning calorimetry (DSC) and thermogravimetric analysis (TGA) and are summarized in Table 4. A melting temperature (Tₘ) from the DSC scans in the range of 30-300° C. was not observed, so a melting point measuring machine was used to observe the melting point (Tₘ) whereas the glass transition temeperatures (T_{g}) were observed at 100 and 130° C. The onset decomposition temperatures (T_{d}) of the compounds were high (greater than 415° C.), which shows their thermal robustness. These results suggest that changing the attached position of the pyridine to the spiro-structure can lead to a significant increase of the thermal stability.

### Example 26

### Electrochemical Properties of 2,7-DPySDF and 3,6-DPySDF

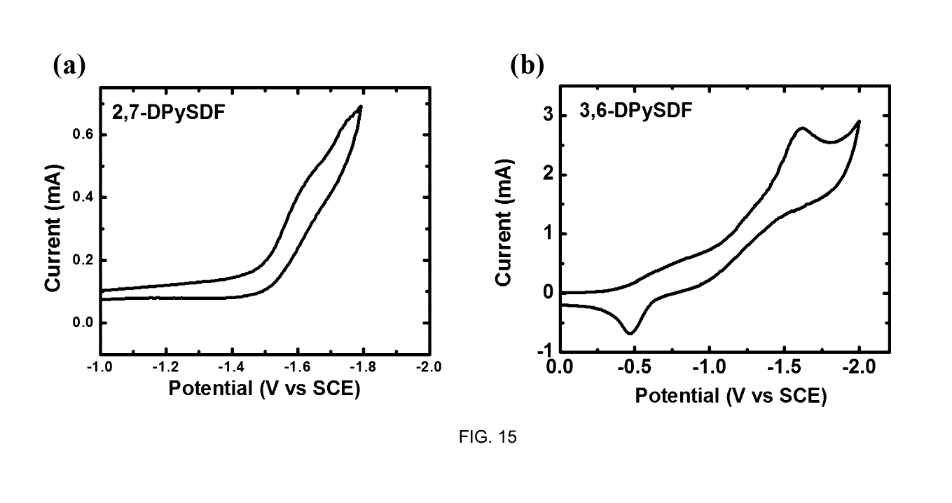

The HOMO/LUMO energy levels of 2,7-DPySDF and 3,6-DPySDF were estimated by cyclic voltammetry (CV) and absorption edge of the UV-Vis spectrum. The cyclic voltamogramms are shown in FIG. 15. The LUMO levels of 2,7-DPySDF and 3,6-DPySDF were estimated from the onset reduction potential of CV, giving LUMO levels of −2.61 eV and −2.71 eV, respectively. The HOMO levels of the 2,7-DPySDF and 3,6-DPySDF were −6.01 eV and −6.24 eV, estimated from the optical band gap. It is believed that the HOMO and LUMO levels of both materials are suitable for facile electron-injection. 3,6-DPySDF showed large optical band gaps (3.53 eV), high lying LUMO energy levels with high triplet energy (3.17 eV). The results demonstrate that these materials are promising for high-performance blue PhOLEDs.

### Example 27

### Device Performance

The use of the compounds of the present invention as the electron-transport layers (ETLs) of blue phosphorescent organic light-emitting diodes (PhOLEDs) was evaluated. To verify the effectiveness of the compounds as ETLs, the following set of blue PhOLEDs were fabricated using a PVK-based emission layer (EML) doped with triplet emitter:

Device I without ETL: ITO/PEDOT:PSS/Blue EML/LiF/Al;

Device II with 3DPySDP ETL: ITO/PEDOT:PSS/Blue EML/3DPySDP (10 nm)LiF/Al;

Device III with 4DPySDP ETL: ITO/PEDOT:PSS/Blue EML/4DPySDP (10 nm)/LiF/Al;

Device IV with 2,7-DPySDF ETL: ITO/PEDOT:PSS/Blue EML/2,7-DPySDF (10 nm)/LiF/Al; Device V with 3,6-DPySDF ETL: ITO/PEDOT:PSS/Blue EML/3,6-DPySDF (10 nm)/LiF/Al;

Device VI with 2PySDP ETL: ITO/PEDOT:PSS/Blue EML/2PySDP (10 nm)/LiF/Al;

Device VII with 3PySDP ETL: ITO/PEDOT:PSS/Blue EML/3PySDP (10 nm)/LiF/Al;

Device VIII with 4PySDP ETL: ITO/PEDOT:PSS/Blue EML/4PySDP (10 nm)/LiF/Al;

Device IX with PSDP ETL: ITO/PEDOT:PSS/Blue EML/PSDP (10 nm)/LiF/Al; and

Device X with DPSDP ETL: ITO/PEDOT:PSS/Blue EML/DPSDP (10 nm)/LiF/Al.

Fabrication of Blue PhOLEDs:

The phosphorescent emission layer (EML) consisted of a blend of PVK and OXD-7 (PVK:OXD-7=60:40, wt/wt) as a host and 10.0 wt % Flrpic as the blue dopant. A solution of Clevios P VP Al 4083 PEDOT:PSS (Heraeus) was used as received. The PEDOT:PSS solution was spin-coated to make a 30-nm hole-injection layer onto pre-cleaned ITO glass. Then the film was annealed at 150° C. under vacuum to remove residual water. The 70-nm polymer EML was obtained by spin coating of the PVK:OXD-7:Flrpic blend in chlorobenzene onto the PEDOT:PSS layer and vacuum dried at 100° C. Each of the dibenzosuberane-based electron transport layers (ETLs) were vacuum-deposited to form 15-nm thin films followed by deposition of 1-nm LiF and 100-nm Al cathode without breaking the vacuum.

Characterization of Blue PhOLEDs:

Film thickness was measured by an Alpha-Step 500 profilometer (KLA-Tencor, San Jose, Calif.) and also confirmed by Atomic Force Microscopy (AFM). Electroluminescence (EL) spectra were obtained using the same spectrofluorimeter described above. Current-voltage (J-V) characteristics of the PhOLEDs were measured by using a HP4155A semiconductor parameter analyzer (Yokogawa Hewlett-Packard, Tokyo). The luminance (brightness) was simultaneously measured by using a model 370 optometer (UDT Instruments, Baltimore, Md.) equipped with a calibrated luminance sensor head (Model 211) and a 5× objective lens. The device external quantum efficiencies (EQEs) were calculated from the forward viewing luminance, current density and EL spectrum assuming a Lambertian distribution. All the device fabrication and device characterization steps were carried out under ambient laboratory condition.

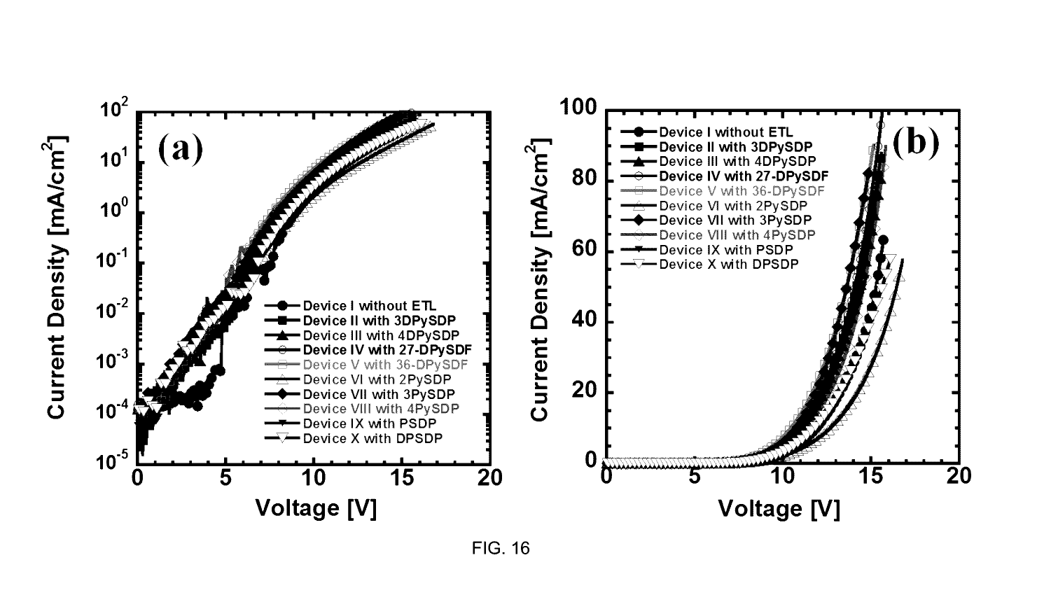

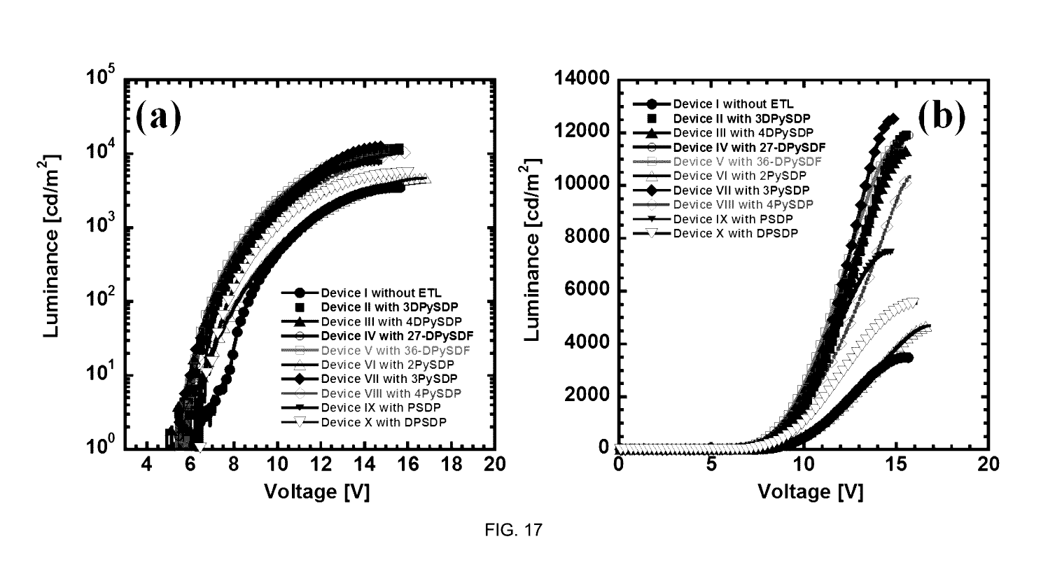

The current density-voltage (J-V) characteristics are shown in log and linear scales in FIG. 16. The current densities of the blue PhOLEDs with dibenzosuberane ETLs increased compared to the device without ETL, except the devices with 2PySDP (device VI) and DPSDP (device X). The luminance-voltage (L-V) characteristics of the PhOLEDs are shown in FIG. 17. The turn-on voltage of the PhOLEDs with dibenzosuberane ETLs were all reduced (5.4-5.8 V) compared to the device without ETL (6.3 V). Blue PhOLEDs with 3DPySDP and 4DPySDP ETLs showed significantly increased brightness of 11920 and 11350 cd/m², respectively. The brightness of the blue PhOLED with dibenzosuberane-based ETL all showed increased brightness compared to the device without ETL (˜3500 cd/m²). However, PhOLEDs with 2PySDP (device VI) and DPSDP (device X) exhibited decreased brightness compared to other devices with dibenzosuberane-based ETLs.

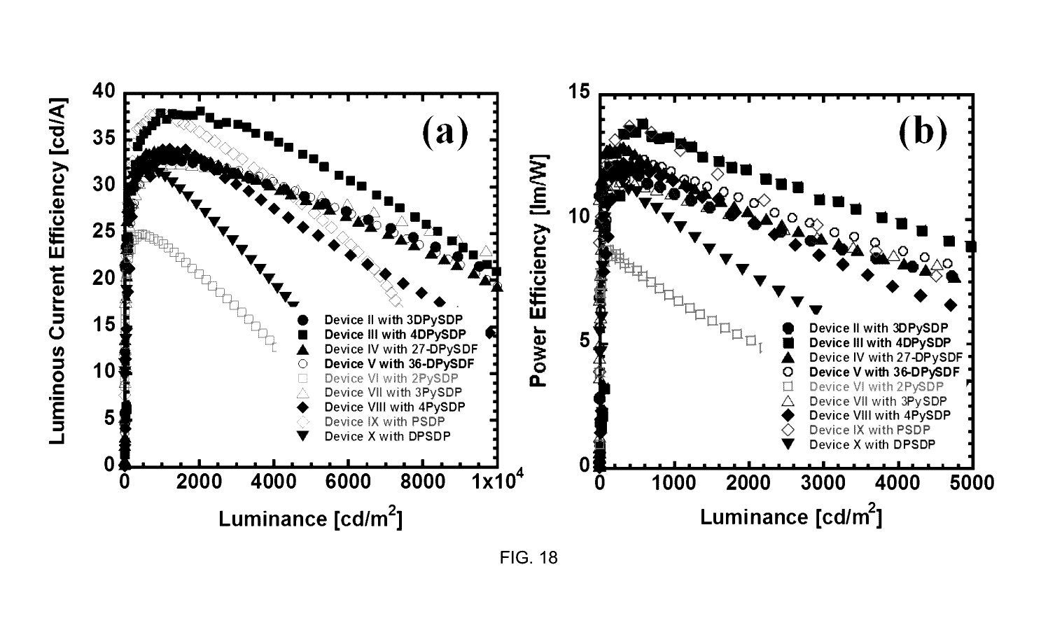

The blue PhOLEDs with dibenzosuberane-based ETLs showed significantly increased efficiency compared to the device without ETL. Luminous efficiency versus luminance and power efficiency versus luminance plots are shown in FIG. 18.

The luminous efficiency (LE) value of the PhOLED with 4DPySDP ETL (device III) showed the highest LE value of 38.1 cd/A at 2030 cd/m² and power efficiency (PE)=13.9 lm/W with an EQE of 20.0%, significantly higher compared to the device without ETL (16.3 cd/A at 600 cd/m² and 5.4 lm/W). PhOLED with PSDP also showed high LE (37.8 cd/A) and PE (14.0 lm/W) values with an EQE of 19.8% (device IX). However, the device showed roll-off of efficiencies with increased luminance. All device performance of blue PhOLEDs with new dibenzosuberane-based materials is summarized in Table 5.

TABLE 5
Device chracteristics of PhOLEDs with dibenzosuberane-based
materials. [a]

| | | | Drive | Current | | Device efficiency |
|---|---|---|---|---|---|---|
| | | Vₒₙ [b] | voltage | density | Luminance | [cd/A, lm/W, |
| Device | ETL | [V] | [V] | [mA/cm²] | [cd/m²] | (% EQE)] |
| Device I | None | 6.3 | 16.4 | 63.3 | 3480 | 5.5, 1.1, (2.8) |
| | | | 9.9 | 2.5 | 600 | 16.3, 5.4, (8.5) |
| Device II | 3DPySDP | 5.4 | 15.6 | 88.0 | 11920 | 13.5, 2.7, (7.1) |
| | | | 9.5 | 3.3 | 1090 | 32.9, 12.2, (17.2) |
| Device III | 4DPySDP | 5.4 | 15.6 | 80.6 | 11350 | 14.1, 2.8, (7.4) |
| | | | 9.9 | 5.3 | 2030 | 38.1, 13.9, (20.0) |
| Device IV | 2,7-DPySDF | 5.5 | 15.7 | 102.3 | 11920 | 11.6, 2.3, (6.1) |
| | | | 8.8 | 2.4 | 760 | 33.9, 13.0, (17.7) |
| Device V | 3,6-DPySDF | 5.5 | 15.2 | 90.3 | 11500 | 12.8, 2.6, (6.7) |
| | | | 8.2 | 1.7 | 570 | 33.1, 12.5, (17.4) |
| Device VI | 2PySDP | 5.8 | 16.8 | 57.8 | 4700 | 8.1, 1.5, (4.2) |
| | | | 9.8 | 1.8 | 460 | 25.0, 8.7, (13.1) |
| Device VII | 3PySDP | 5.4 | 13.6 | 187.8 | 12500 | 15.0, 3.2, (7.9) |
| | | | 9.8 | 4.8 | 1560 | 32.5, 11.5, (17.0) |
| Device VIII | 4PySDP | 5.4 | 15.9 | 90.0 | 10370 | 11.5, 2.3, (6.0) |
| | | | 9.1 | 2.2 | 880 | 34.3, 11.7, (18.0) |
| Device IX | PSDP | 6.0 | 14.8 | 53.3 | 7480 | 14.0, 3.0, (7.3) |
| | | | 9.2 | 2.6 | 1000 | 37.8, 14.0, (19.8) |
| Device X | DPSDP | 6.1 | 16.1 | 57.9 | 5540 | 9.6, 1.9, (5.0) |
| | | | 9.2 | 1.6 | 530 | 32.3, 11.7, (16.9) |

[a] Values in italic correspond to those at maximum device efficiencies.
[b] Turn-on voltage (at brightness of 1 cd/m²).

The blue PhOLEDs with dibenzosuberane-based ETM showed improved device performances. These results demonstrate that these new dibenzosuberane-based compounds are promising electron transport material with good exciton blocking ability in PhOLEDs.

1. A compound having the structure represented by formula (I): 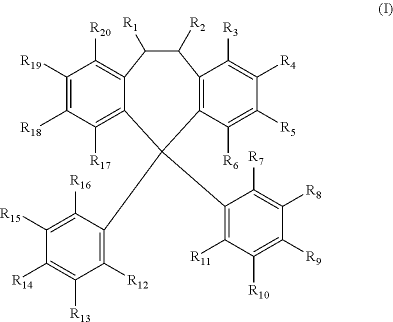
wherein
R₁, R₂, R₈, R₉, R₁₄, and R₁₅ are each, independently, a substituent selected from H, halo, cyano, hydroxyl, alkyl, alkenyl, alkynyl, heteroalkyl, heteroalkenyl, heteroalkynyl, alkoxyl, 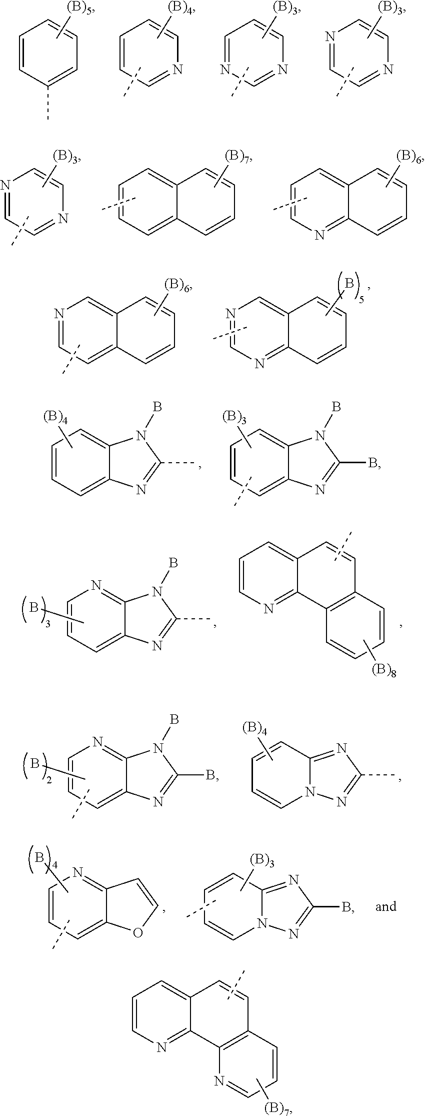
wherein each occurrence of B is, independently, a substituent selected from H, alkyl, alkenyl, alkynyl, heteroalkyl, heteroalkenyl, heteroalkynyl, aryl, heterocyclyl, and heteroaryl;
R₃, R₄, R₅, R₆, R₇, R₁₀, R₁₃, R₁₆, R₁₇, R₁₈, R₁₉, and R₂₀ are each, independently, a substituent selected from H, cyano, hydroxyl, alkyl, alkenyl, alkynyl, heteroalkyl, heteroalkenyl, heteroalkynyl, and alkoxyl;
R₁₁ and R₁₂ are each, independently, a substituent selected from H, cyano, hydroxyl, alkyl, alkenyl, alkynyl, heteroalkyl, heteroalkenyl, heteroalkynyl, and alkoxyl;
or R₁₁ and R₁₂ together form a bond;
wherein each substituent may optionally be further substituted; and
wherein at least one of R₁, R₂, R₈, R₉, R₁₄, and R₁₅ is not a substituent selected from H, cyano, hydroxyl, alkyl, alkenyl, alkynyl, heteroalkyl, heteroalkenyl, heteroalkynyl, and alkoxyl.

2. The compound according to claim 1, wherein
R₁₁ and R₁₂ are each, independently, a substituent selected from H, cyano, hydroxyl, alkyl, alkenyl, alkynyl, heteroalkyl, heteroalkenyl, heteroalkynyl, and alkoxyl.

3. The compound according to claim 1, wherein
R₁₁ and R₁₂ together form a bond.

4. (canceled)

5. (canceled)

6. The compound according to claim 1, wherein
R₈, R₉, R₁₄, and R₁₅, are each, independently, a substituent selected from H, halo,

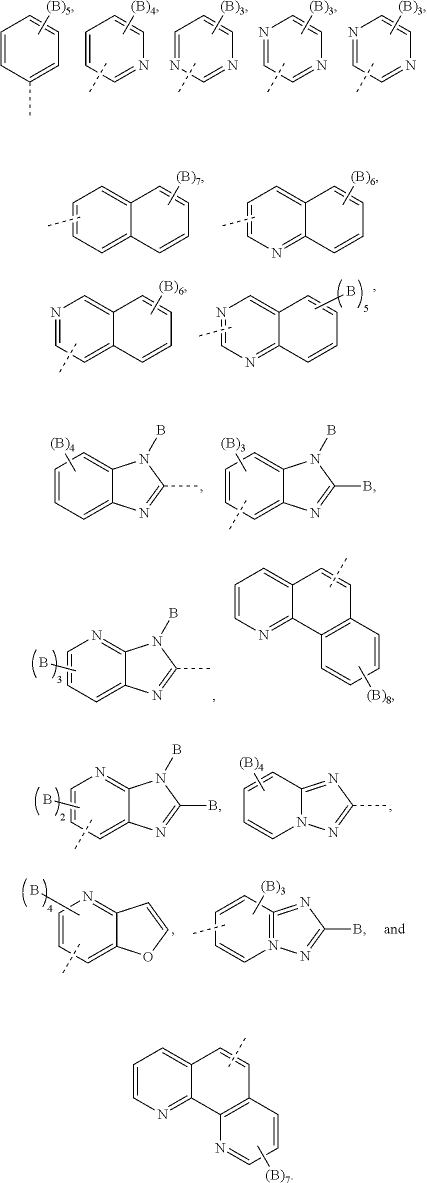

7. The compound according to claim 1, wherein the compound has the structure

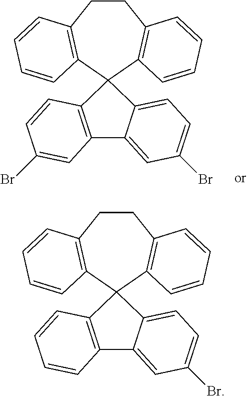 [SMILES: Brc1ccc2c(c1)-c1cc(Br)ccc1C21c2ccccc2CCc2ccccc21 ; "or" ; Brc1ccc2c(c1)-c1ccccc1C21c2ccccc2CCc2ccccc21]

8. The compound according to claim 1, wherein the compound has the structure 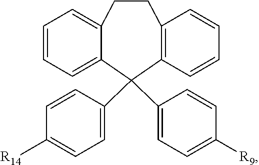
wherein R₉ and R₁₄ are each, independently, selected from H, 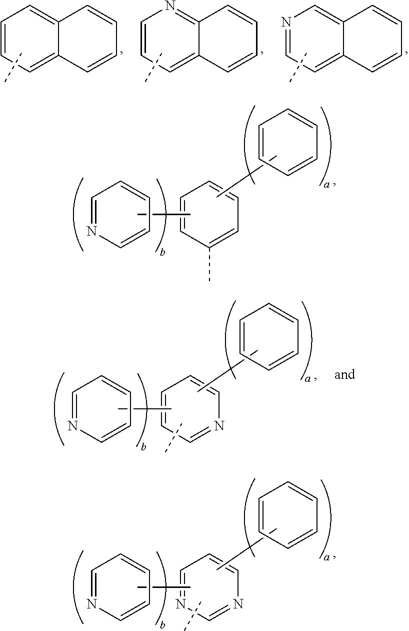
wherein a+b=0, 1, or 2.

9. The compound according to claim 1, wherein the compound has the structure

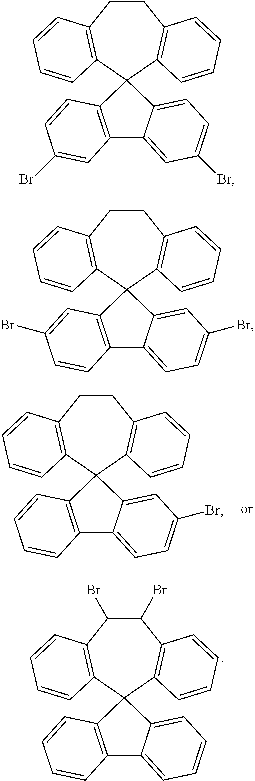

10. The compound according to claim 1, wherein the compound has the structure 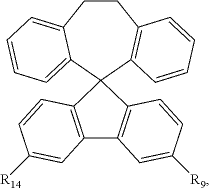
wherein R₉ and R₁₄ are each, independently, selected from H, 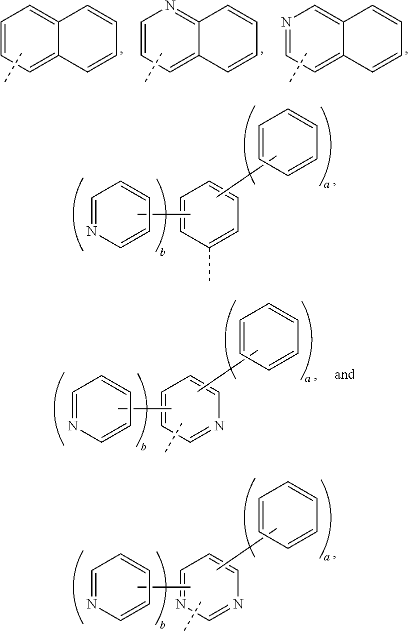
wherein a+b=0, 1, or 2.

11. The compound according to claim 1, wherein the compound has the structure 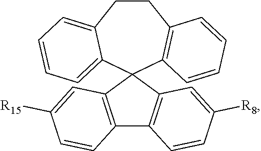
wherein R₈ and R₁₅ are each, independently, selected from H, 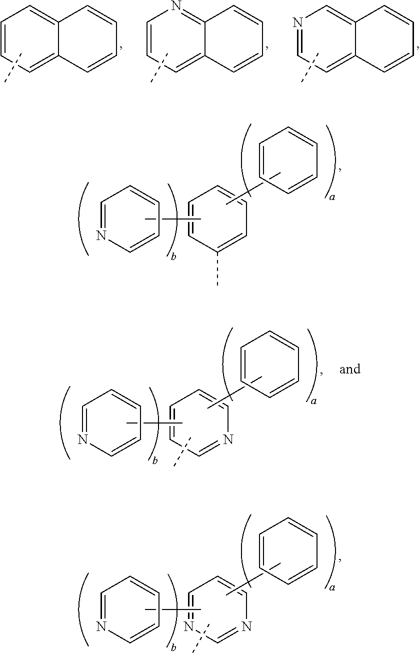
wherein a+b=0, 1, or 2.

12. The compound according to claim 1, wherein the compound has the structure 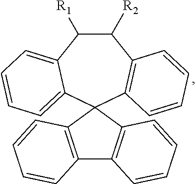
wherein R₁ and R₂ are each, independently, selected from H, 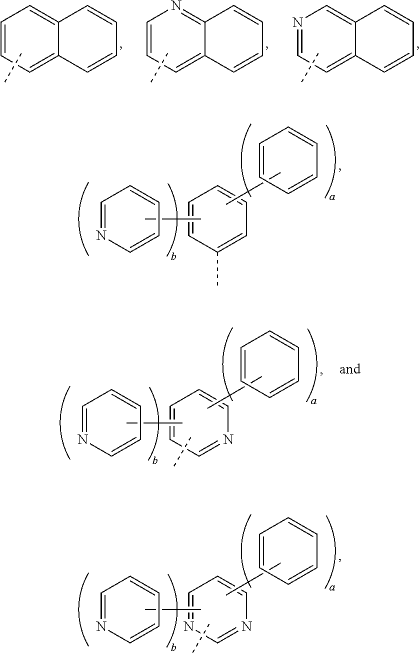
wherein a+b=0, 1, or 2.

13. A process for making a compound according to claim 1, the process comprising:
(1a) contacting a compound having the structure 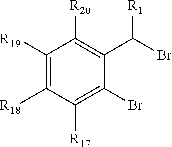
with a compound having the structure 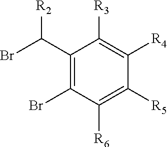
in the presence of a compound R′—Li, wherein R′ is (C₁-C₅)alkyl, to form a compound having the structure 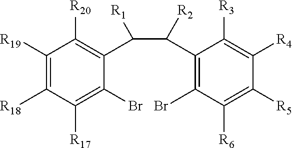
wherein
R₁ and R₂ are each, independently, a substituent selected from H, halo, cyano, hydroxyl, alkyl, alkenyl, alkynyl, heteroalkyl, heteroalkenyl, heteroalkynyl, alkoxyl, 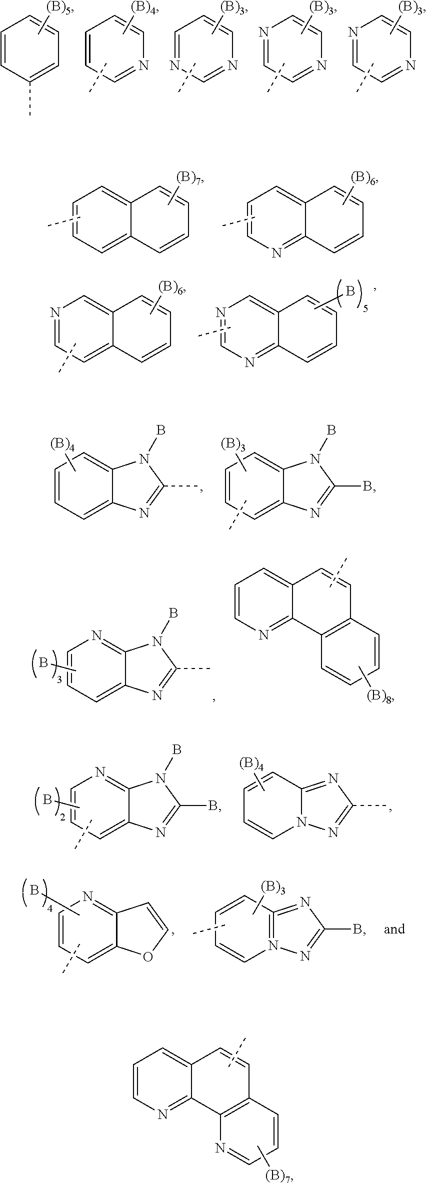
wherein each occurrence of B is, independently, a substituent selected from H, alkyl, alkenyl, alkynyl, heteroalkyl, heteroalkenyl, heteroalkynyl, aryl, heterocyclyl, and heteroaryl;
R₃, R₄, R₅, R₆, R₁₇, R₁₈, R₁₉, and R₂₀ are each, independently, a substituent selected from H, cyano, hydroxyl, alkyl, alkenyl, alkynyl, heteroalkyl, heteroalkenyl, heteroalkynyl, and alkoxyl;
(1b) contacting the compound formed in step (1a) with a compound having the structure 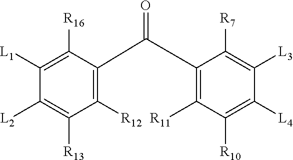
in the presence of a compound R″—Li, wherein R″ is (C₁-C₅)alkyl, to form a compound having the structure 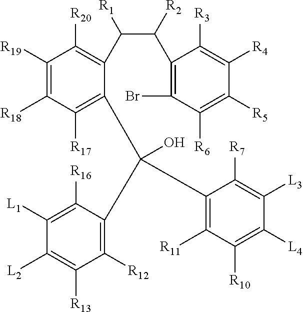
wherein
L₁, L₂, L₃, and L₄ are each, independently, a substituent selected from H, halo, trifluoromethanesulfonyl, cyano, hydroxyl, alkyl, alkenyl, alkynyl, heteroalkyl, heteroalkenyl, heteroalkynyl, and alkoxyl;
R₇, R₁₀, R₁₃, and R₁₆ are each, independently, a substituent selected from H, cyano, hydroxyl, alkyl, alkenyl, alkynyl, heteroalkyl, heteroalkenyl, heteroalkynyl, and alkoxyl;
R₁₁ and R₁₂ are each, independently, a substituent selected from H, cyano, hydroxyl, alkyl, alkenyl, alkynyl, heteroalkyl, heteroalkenyl, heteroalkynyl, and alkoxyl;
or R₁₁ and R₁₂ together form a bond; and
wherein each substituent may optionally be further substituted.

14. The process according to claim 13, further comprising:
(1c) contacting the compound formed in step (1b) with an acid to form a compound having the structure

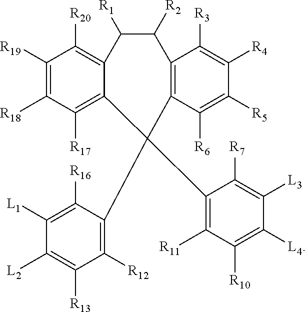

15. (canceled)

16. The process according to claim 15, further comprising:
(1d) contacting the compound formed in step (1c) with a compound R′″—Z, wherein R′″ is selected from 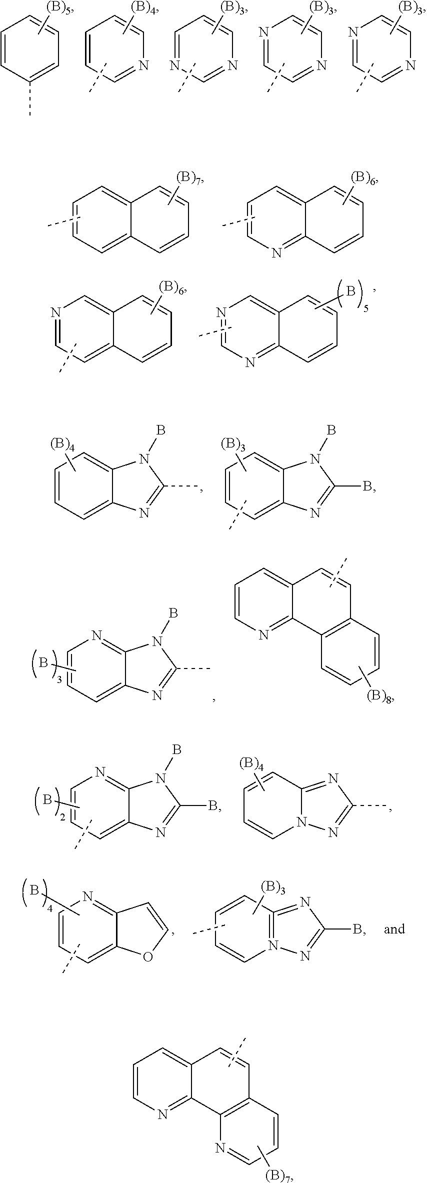
wherein each occurrence of B is, independently, a substituent selected from H, alkyl, alkenyl, alkynyl, heteroalkyl, heteroalkenyl, heteroalkynyl, aryl, heterocyclyl, and heteroaryl; and
Z is —B(OH)₂ or —ZnBr;
in the presence of a palladium catalyst to form a compound having the structure 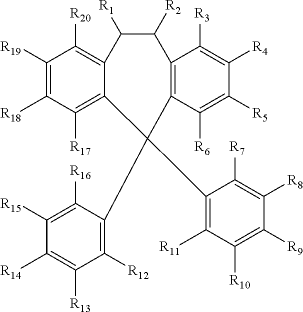
wherein
R₈, R₉ and R₁₄, and R₁₅ are each, independently, a substituent selected from H,

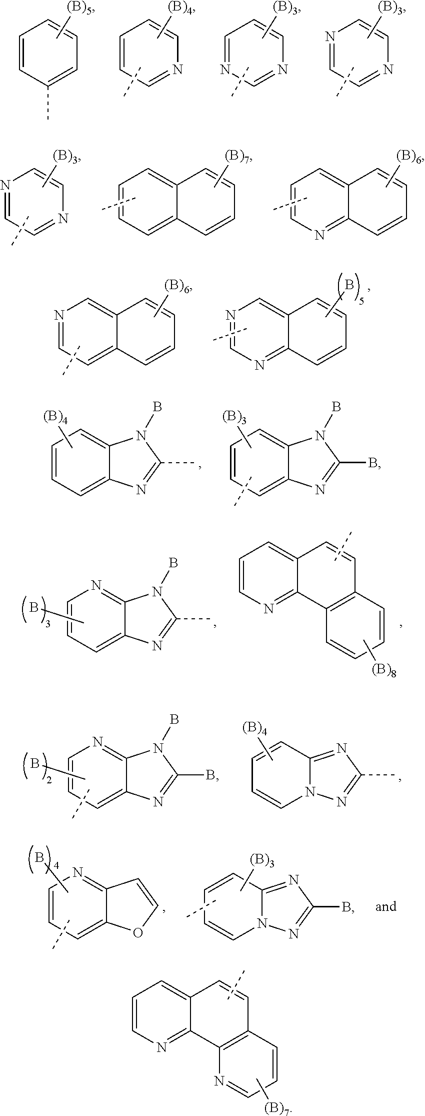

17. A process for making a compound according to claim 1, the process comprising:
(2a) contacting a compound having the structure 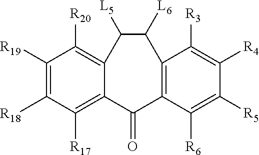
with a compound having the structure 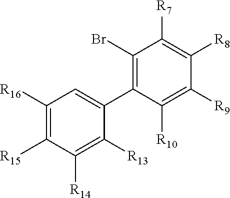
in the presence of R′—Li, wherein R′ is (C₁-C₅)alkyl, to form a compound having the structure 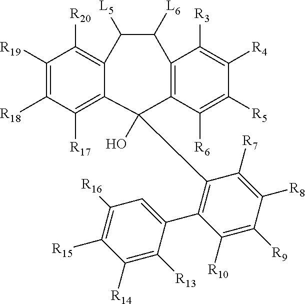
L₅ and L₆ are each, independently, a substituent selected from H, halo, trifluoromethanesulfonyl, cyano, hydroxyl, alkyl, alkenyl, alkynyl, heteroalkyl, heteroalkenyl, heteroalkynyl, alkoxyl;
R₈, R₉, R₁₄, and R₁₅ are each, independently, a substituent selected from H, halo, cyano, hydroxyl, alkyl, alkenyl, alkynyl, heteroalkyl, heteroalkenyl, heteroalkynyl, alkoxyl, 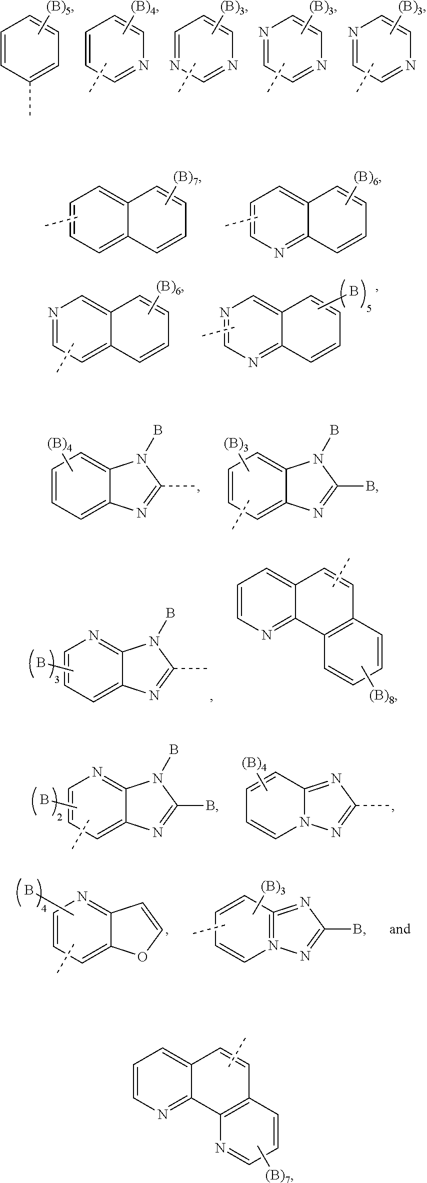
wherein each occurrence of B is, independently, a substituent selected from H, alkyl, alkenyl, alkynyl, heteroalkyl, heteroalkenyl, heteroalkynyl, aryl, heterocyclyl, and heteroaryl;
R₃, R₄, R₅, R₆, R₇, R₁₀, R₁₃, R₁₆, R₁₇, R₁₈, R₁₉, and R₂₀ are each, independently, a substituent selected from H, cyano, hydroxyl, alkyl, alkenyl, alkynyl, heteroalkyl, heteroalkenyl, heteroalkynyl, and alkoxyl.

18. The process according to claim 17, further comprising:
(2b) contacting the compound formed in step (2a) with an acid to form a compound having the structure

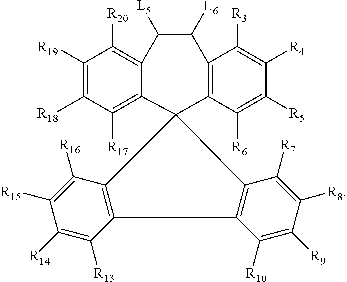

19.-24. (canceled)

25. A composition comprising at least one compound according to claim 1.

26. An ink composition comprising at least one liquid carrier and at least one compound according to claim 1.

27. A device comprising one or several layers comprising at least one compound according to claim 1.

28. The device according to claim 27, wherein the device is a light emitting diode, a field-effect transistor, or a photovoltaic cell

29. The device of claim 28 wherein the device is an organic light emitting diode.

30. (canceled)

31. (canceled)
